(19) United States
(12) Patent Application Publication
(10) Pub. No.: US 2024/0065767 A1
(43) Pub. Date: Feb. 29, 2024

(54) SYSTEMS AND METHODS FOR GENERATING MULTIPLE PATIENT-SPECIFIC SURGICAL PLANS AND MANUFACTURING PATIENT-SPECIFIC IMPLANTS

(71) Applicant: Carlsmed, Inc., Carlsbad, CA (US)

(72) Inventors: Michael J. Cordonnier, Carlsbad, CA (US); Niall Patrick Casey, Carlsbad, CA (US); Syed Shariq Hussain, Vista, CA (US)

(21) Appl. No.: 18/455,881

(22) Filed: Aug. 25, 2023

Related U.S. Application Data
(60) Provisional application No. 63/401,429, filed on Aug. 26, 2022.

(51) Int. Cl. A61B 34/10; A61F 2/44; A61F 2/46

(52) U.S. Cl. CPC A61B 34/10; A61F 2/4455; A61F 2/4611; A61B 2034/104; A61B 2034/105; A61F 2002/4633

(57) ABSTRACT

Systems and methods for planning and implementing medical procedures and/or devices are disclosed. For example, many embodiments include generating multiple patient-specific surgical plans, with each of the multiple patient-specific surgical plans providing a different recommended treatment protocol for a patient. Each patient-specific surgical plan can also provide predicted post-operative data associated with the treatment protocol.

### CROSS-REFERENCE TO RELATED APPLICATION(S)

This application claims priority to and the benefit of U.S. Provisional Patent Application No. 63/401,429, filed Aug. 26, 2022, which is hereby incorporated by reference in its entirety.

### TECHNICAL FIELD

The present disclosure is generally related to designing and implementing medical care, and more particularly to systems and methods for designing and implementing patient-specific surgical procedures and/or medical devices.

### BACKGROUND

Surgical procedures to implant orthopedic implants are used to correct numerous different maladies in a variety of contexts, including spine surgery, hand surgery, shoulder and elbow surgery, total joint reconstruction (arthroplasty), skull reconstruction, pediatric orthopedics, foot and ankle surgery, musculoskeletal oncology, surgical sports medicine, and orthopedic trauma. Spine surgery itself may encompass a variety of procedures and targets, such as one or more of the cervical spine, thoracic spine, lumbar spine, or sacrum, and may be performed to treat a deformity or degeneration of the spine and/or related back pain, leg pain, or other body pain. Common spinal deformities that may be treated using an orthopedic implant include irregular spinal curvature such as scoliosis, lordosis, or kyphosis (hyper- or hypo-), and irregular spinal displacement (e.g., spondylolisthesis). Other spinal disorders that can be treated using an orthopedic implant include osteoarthritis, lumbar degenerative disc disease or cervical degenerative disc disease, lumbar spinal stenosis, and cervical spinal stenosis.

### BRIEF DESCRIPTION OF THE DRAWINGS

The accompanying drawings illustrate various embodiments of systems, methods, and embodiments of various other aspects of the disclosure. Any person with ordinary skill in the art will appreciate that the illustrated element boundaries (e.g., boxes, groups of boxes, or other shapes) in the figures represent one example of the boundaries. It may be that in some examples one element may be designed as multiple elements or that multiple elements may be designed as one element. In some examples, an element shown as an internal component of one element may be implemented as an external component in another, and vice versa. Furthermore, elements may not be drawn to scale. Non-limiting and non-exhaustive descriptions are described with reference to the following drawings. The components in the figures are not necessarily to scale, emphasis instead being placed upon illustrating principles.

FIG. 1 is a network connection diagram illustrating a system for providing patient-specific medical care in accordance with embodiments of the present technology.

FIG. 2 illustrates a computing device suitable for use in connection with the system of FIG. 1 in accordance with embodiments of the present technology.

FIG. 3A is a flow diagram illustrating a method for providing patient-specific medical care in accordance with embodiments of the present technology.

FIG. 3B is a flow diagram illustrating another method for providing patient-specific medical care in accordance with embodiments of the present technology.

FIGS. 4A and 4B illustrate a representative surgical plan generated in accordance with embodiments of the present technology.

FIG. 5 illustrates two representative surgical plans generated in accordance with embodiments of the present technology.

FIG. 6 is a flow diagram illustrating another method for providing patient-specific medical care in accordance with embodiments of the present technology.

FIGS. 7A-7C illustrate representative data sets that may be used and/or generated in connection with the methods described herein, in accordance with embodiments of the present technology. FIG. 4A illustrates a patient data set. FIG. 4B illustrates a plurality of reference patient data sets. FIG. 4C illustrates similarity scores and outcome scores for the reference patient data sets of FIG. 4B.

FIG. 8 is a flow diagram illustrating another method for providing patient-specific medical care in accordance with embodiments of the present technology.

FIG. 9 is a partially schematic illustration of an operative setup and associated computing systems for providing patient-specific medical care in accordance with embodiments of the present technology.

FIGS. 10A and 10B illustrate a representative patient-specific implant that can be used and/or generated in connection with the methods described herein in accordance with embodiments of the present technology.

FIG. 11 illustrates a segment of a patient's spine after several patient-specific implants have been implanted therein in accordance with embodiments of the present technology.

### DETAILED DESCRIPTION

The present technology is directed to systems and methods for planning and implementing medical procedures and/or devices. For example, in many of the embodiments disclosed herein, a method of providing medical care includes generating multiple patient-specific surgical plans. Each of the multiple patient-specific surgical plans can provide a different recommended treatment protocol, such as a different surgical intervention or surgery location, that could be performed on the patient. In addition to providing a surgical intervention and surgery location, each patient-specific surgical plan can also provide predicted post-operative data associated with the corresponding surgical intervention and/or surgical location. For example, each surgical plan may include a virtual model of predicted post-operative patient anatomy if the surgical plan were to be performed. Each surgical plan may also include one or more patient metrics associated with the predicted post-operative patient anatomy, or other predicted post-operative patient data.

Without being bound by theory, generating, designing, and/or modeling multiple surgical plans that include predicted post-operative outcomes is expected to be beneficial because it permits a surgeon or other healthcare provider to review and select between several different surgical plans based at least in part on predicted outcomes, patient preferences, surgeon preferences, or the like. For example, this may be beneficial because it will enable a surgeon to select a surgical plan with the predicted outcome that is best suited for the patient's lifestyle, activity level, age, weight, comorbidities, etc. This may also be beneficial because it will enable the surgeon to select a surgical plan that incorporates surgical techniques and procedures that the surgeon is experienced with.

Embodiments of the present disclosure will be described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

The words “comprising,” “having,” “containing,” and “including,” and other forms thereof, are intended to be equivalent in meaning and be open ended in that an item or items following any one of these words is not meant to be an exhaustive listing of such item or items, or meant to be limited to only the listed item or items.

As used herein and in the appended claims, the singular forms “a,” “an,” and “the” include plural references unless the context clearly dictates otherwise.

Although the disclosure herein primarily describes systems and methods for treatment planning in the context of orthopedic surgery, the technology may be applied equally to medical treatment and devices in other fields (e.g., other types of surgical practice). Additionally, although many embodiments herein describe systems and methods with respect to implanted devices, the technology may be applied equally to other types of medical devices (e.g., non-implanted devices).

The headings are provided for convenience only and should not be used to interpret the scope of the present technology.

### A. Select Embodiments of Systems for Designing Patient-Specific Surgical Plans and Patient-Specific Implants

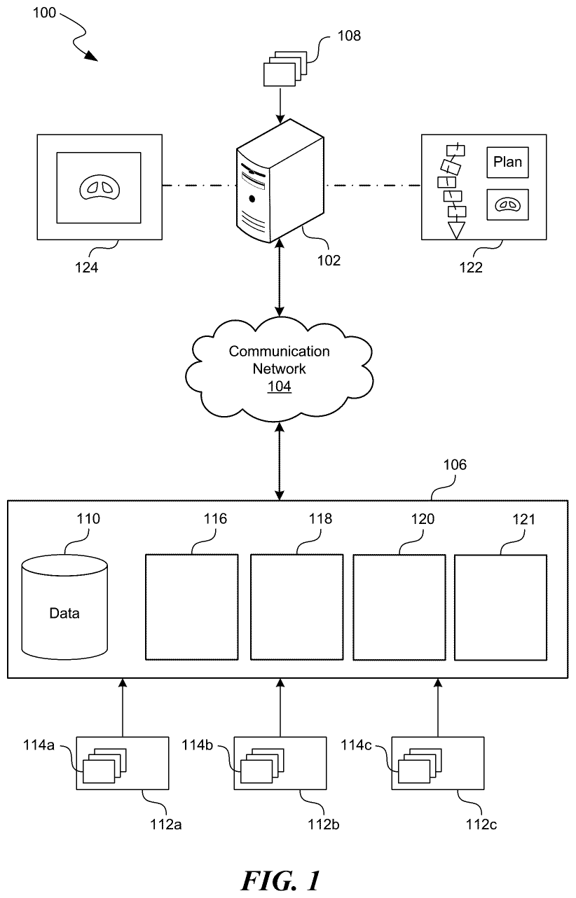

FIG. 1 is a network connection diagram illustrating a computing system 100 for providing patient-specific medical care, according embodiments of the present technology. As described in further detail herein, the system 100 is configured to generate a medical treatment plan for a patient. In some embodiments, the system 100 is configured to generate a medical treatment plan for a patient suffering from an orthopedic or spinal disease or disorder, such as trauma (e.g., fractures), cancer, deformity, degeneration, pain (e.g., back pain, leg pain), irregular spinal curvature (e.g., scoliosis, lordosis, kyphosis), irregular spinal displacement (e.g., spondylolisthesis, lateral displacement axial displacement), osteoarthritis, lumbar degenerative disc disease, cervical degenerative disc disease, lumbar spinal stenosis, or cervical spinal stenosis, or a combination thereof. The medical treatment plan can include surgical information, technology recommendations (e.g., device and/or instrument recommendations), and/or medical device designs. For example, the medical treatment plan can include at least surgical procedure (e.g., a surgical procedure or intervention) and/or at least one medical device (e.g., an implanted medical device (also referred to herein as an “implant” or “implanted device”) or implant delivery instrument). In some embodiments, the medical treatment plan is therefore also referred to as a “surgical plan,” a “patient-specific surgical plan,” a “patient-specific treatment plan,” or the like.

In some embodiments, the system 100 generates a medical treatment plan that is customized for a particular patient or group of patients, also referred to herein as a “patient-specific” or “personalized” treatment or surgical plan. The patient-specific surgical plan can include at least one patient-specific surgical procedure and/or at least one patient-specific medical device that are designed and/or optimized for the patient's particular characteristics (e.g., condition, anatomy, pathology, condition, medical history). For example, the patient-specific medical device can be designed and manufactured specifically for the particular patient, rather than being an off-the-shelf device. However, it shall be appreciated that a patient-specific surgical plan can also include aspects that are not customized for the particular patient. For example, a patient-specific or personalized surgical procedure can include one or more instructions, portions, steps, etc. that are non-patient-specific. Likewise, a patient-specific or personalized medical device can include one or more components that are non-patient-specific, and/or can be used with an instrument or tool that is non-patient-specific. Personalized implant designs can be used to manufacture or select patient-specific technologies, including medical devices, instruments, and/or surgical kits. For example, a personalized surgical kit can include one or more patient-specific devices, patient-specific instruments, non-patient-specific technology (e.g., standard instruments, devices, etc.), instructions for use, patient-specific treatment plan information, or a combination thereof.

The system 100 includes a client computing device 102, which can be a user device, such as a smart phone, mobile device, laptop, desktop, personal computer, tablet, phablet, or other such devices known in the art. As discussed further herein, the client computing device 102 can include one or more processors, and memory storing instructions executable by the one or more processors to perform the methods described herein. The client computing device 102 can be associated with a healthcare provider (e.g., a surgeon) that is treating the patient. Although FIG. 1 illustrates a single client computing device 102, in alternative embodiments, the client computing device 102 can instead be implemented as a client computing system encompassing a plurality of computing devices, such that the operations described herein with respect to the client computing device 102 can instead be performed by the computing system and/or the plurality of computing devices.

The client computing device 102 is configured to receive a patient data set 108 associated with a patient to be treated. The patient data set 108 can include data representative of the patient's condition, anatomy, pathology, medical history, preferences, and/or any other information or parameters relevant to the patient. For example, the patient data set 108 can include medical history, surgical intervention data, treatment outcome data, progress data (e.g., physician notes), patient feedback (e.g., feedback acquired using quality of life questionnaires, surveys), clinical data, provider information (e.g., physician, hospital, surgical team), patient information (e.g., demographics, sex, age, height, weight, type of pathology, occupation, activity level, tissue information, health rating, comorbidities, health related quality of life (HRQL)), vital signs, diagnostic results, medication information, allergies, image data (e.g., camera images, Magnetic Resonance Imaging (MRI) images, ultrasound images, Computerized Aided Tomography (CAT) scan images, Positron Emission Tomography (PET) images, X-Ray images), diagnostic equipment information (e.g., manufacturer, model number, specifications, user-selected settings/configurations, etc.), or the like. In some embodiments, the patient data set 108 includes data representing one or more of patient identification number (ID), age, gender, body mass index (BMI), lumbar lordosis, Cobb angle(s), pelvic incidence, disc height, segment flexibility, bone quality, rotational displacement, and/or treatment level of the spine.

The client computing device 102 is operably connected via a communication network 104 to a server 106, thus allowing for data transfer between the client computing device 102 and the server 106. The communication network 104 may be a wired and/or a wireless network. The communication network 104, if wireless, may be implemented using communication techniques such as Visible Light Communication (VLC), Worldwide Interoperability for Microwave Access (WiMAX), Long term evolution (LTE), Wireless local area network (WLAN), Infrared (IR) communication, Public Switched Telephone Network (PSTN), Radio waves, and/or other communication techniques known in the art.

The server 106, which may also be referred to as a “treatment assistance network” or “prescriptive analytics network,” can include one or more computing devices and/or systems. As discussed further herein, the server 106 can include one or more processors, and memory storing instructions executable by the one or more processors to perform the methods described herein. In some embodiments, the server 106 is implemented as a distributed “cloud” computing system or facility across any suitable combination of hardware and/or virtual computing resources.

The client computing device 102 and server 106 can individually or collectively perform the various methods described herein for providing patient-specific medical care. For example, some or all of the steps of the methods described herein can be performed by the client computing device 102 alone, the server 106 alone, or a combination of the client computing device 102 and the server 106. Thus, although certain operations are described herein with respect to the server 106, it shall be appreciated that these operations can also be performed by the client computing device 102, and vice-versa.

The server 106 includes at least one database 110 configured to store reference data useful for the treatment planning methods described herein. The reference data can include historical and/or clinical data from the same or other patients, data collected from prior surgeries and/or other treatments of patients by the same or other healthcare providers, data relating to medical device designs, data collected from study groups or research groups, data from practice databases, data from academic institutions, data from implant manufacturers or other medical device manufacturers, data from imaging studies, data from simulations, clinical trials, demographic data, treatment data, outcome data, mortality rates, or the like.

In some embodiments, the database 110 includes a plurality of reference patient data sets, each patient reference data set associated with a corresponding reference patient. For example, the reference patient can be a patient that previously received treatment or is currently receiving treatment. Each reference patient data set can include data representative of the corresponding reference patient's condition, anatomy, pathology, medical history, disease progression, preferences, and/or any other information or parameters relevant to the reference patient, such as any of the data described herein with respect to the patient data set 108. In some embodiments, the reference patient data set includes pre-operative data, intra-operative data, and/or post-operative data. For example, a reference patient data set can include data representing one or more of patient ID, age, gender, BMI, lumbar lordosis, Cobb angle(s), pelvic incidence, disc height, segment flexibility, bone quality, rotational displacement, and/or treatment level of the spine. As another example, a reference patient data set can include treatment data regarding at least one surgical procedure performed on the reference patient, such as descriptions of surgical procedures or interventions (e.g., surgical approaches, bony resections, surgical maneuvers, corrective maneuvers, placement of implants or other devices). In some embodiments, the treatment data includes medical device design data for at least one medical device used to treat the reference patient, such as physical properties (e.g., size, shape, volume, material, mass, weight), mechanical properties (e.g., stiffness, strength, modulus, hardness), and/or biological properties (e.g., osteo-integration, cellular adhesion, anti-bacterial properties, anti-viral properties). In yet another example, a reference patient data set can include outcome data representing an outcome of the treatment of the reference patient, such as corrected anatomical metrics, presence of fusion, HRQL, pain level, activity level, return to work, complications, recovery times, efficacy, mortality, and/or follow-up surgeries.

In some embodiments, the server 106 can include accounts with electronic medical records and data for patients. The accounts can be linked to three-dimensional representations, virtual models of patient anatomy, surgical plans, manufacturing data, or other data disclosed herein to, for example, synchronize, update, or otherwise coordinate management of aspects of surgical planning, implant design, implant manufacturing, etc. For example, an account for a patient can include a virtual anatomical model that is dynamically updated and analyzed based on user input (e.g., from surgeons, healthcare providers, implant designers, and/or the patient). The virtual anatomical model is dynamically updated with pre-operative data, intra-operative data, and/or post operative data. The user input can be provided via one or more interactive surgical planners, electronic surgical plans, or the like.

The server 106 can store one or more virtual models that include, for example, repositionable anatomical elements, model data, and user-set controls/constraints. The repositionable anatomical elements can be moved based upon the model data. The model data can include one or more tags, parametric parameters, model constraints, boundary conditions, analysis data (e.g., finite element analysis data), or the like. In some embodiments, the server 106 can analyze virtual anatomical models and/or patient images to identify features, tag features, correlate features, etc. For example, features of anatomical elements can be tagged for measuring or determining metrics. The tagged features can be correlated to one another to generate mappings, including reciprocal changes mappings. A set of virtual models for a patient can have the same tagged features to enable modification of same metrics between the models.

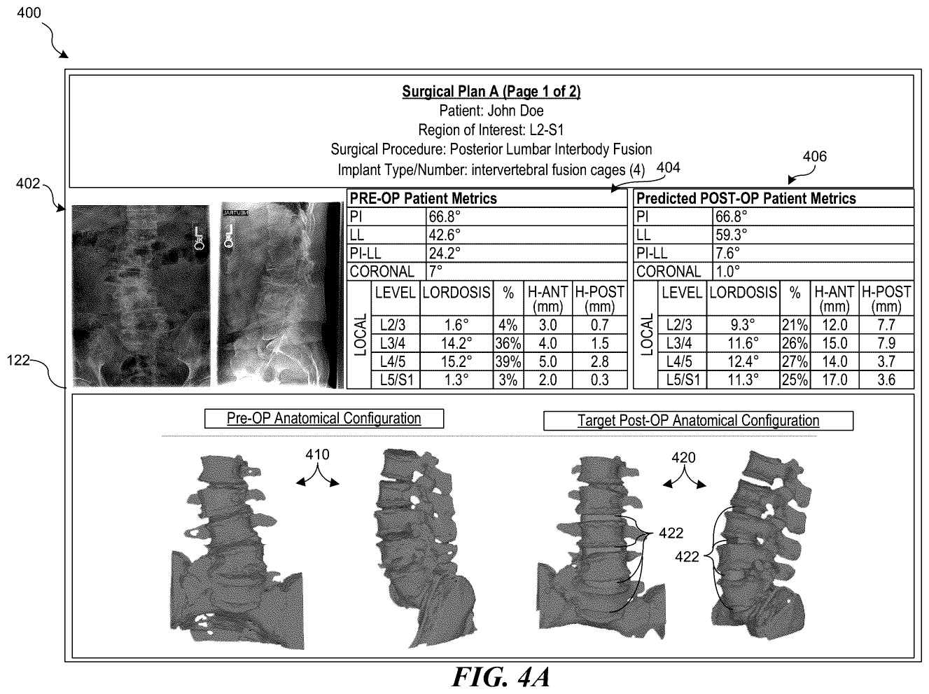

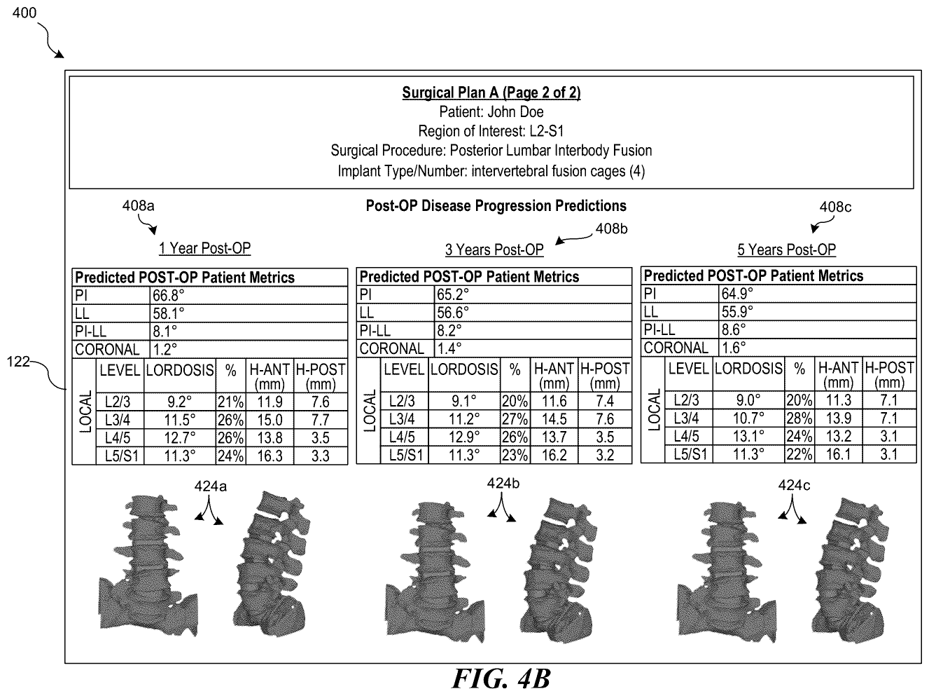

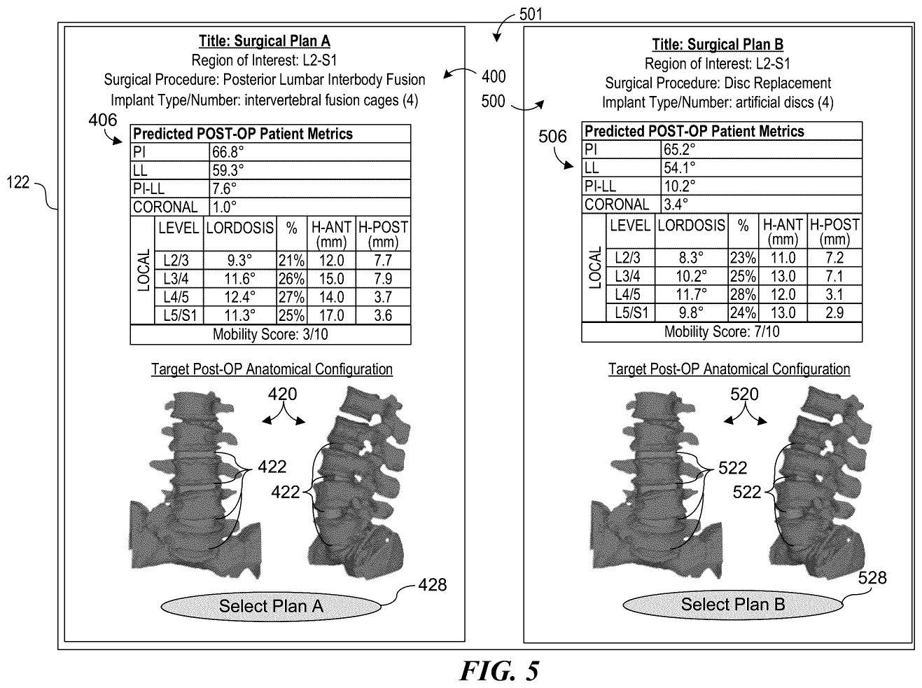

The server 106 can receive user input and can transform the received user input into model data for updating the virtual model. By way of example, a user can input one or more target metrics (e.g., target values for metrics discussed in connection with FIGS. 4A-5). The server 106 can then transform the inputted values for the target metrics into positional relationships (e.g., relative positions) for anatomical elements based on one or more transformation matrices. The transformation matrices can be generated using machine-learning algorithms. The server 106 can then reposition the anatomical elements of the model to meet the inputted values for the tagged features (e.g., distance between tagged vertebral endplates, lordosis relative to tagged features of endplates, etc.). In some embodiments, the server 106 can transform image data into the model data to generate new virtual models, modify virtual models based on the most recent image data, or the like. The server 106 can also extract position data for anatomical features from image data of the patient. The extracted data can then be mapped to tagged features of the anatomical model. The server 106 can determine whether to modify, replace, or otherwise alter the virtual model based on a comparison of positional information from the images to corresponding positional information of the virtual model. This allows for dynamic model updating using different types of data. In some embodiments, the server 106 generates instructions commanding CAD software to perform model updating based on one or more updating triggers, such as detection of new image data for the patient, change in patient status, etc.

In some embodiments, the server 106 receives at least some of the reference patient data sets from a plurality of healthcare provider computing systems (e.g., systems 112a-112c, collectively 112). The server 106 can be connected to the healthcare provider computing systems 112 via one or more communication networks (not shown). Each healthcare provider computing system 112 can be associated with a corresponding healthcare provider (e.g., physician, surgeon, medical clinic, hospital, healthcare network, etc.). Each healthcare provider computing system 112 can include at least one reference patient data set (e.g., reference patient data sets 114a-114c, collectively 114) associated with reference patients treated by the corresponding healthcare provider. The reference patient data sets 114 can include, for example, electronic medical records, electronic health records, biomedical data sets, etc. The reference patient data sets 114 can be received by the server 106 from the healthcare provider computing systems 112 and can be reformatted into different formats for storage in the database 110. Optionally, the reference patient data sets 114 can be processed (e.g., cleaned) to ensure that the represented patient parameters are likely to be useful in the treatment planning methods described herein.

As described in further detail herein, the server 106 can be configured with one or more algorithms that generate patient-specific surgical plan data (e.g., treatment procedures, target anatomical corrections, medical devices, etc.) based on the reference data. In some embodiments, the patient-specific data is generated based on correlations between the patient data set 108 and the reference data. Optionally, the server 106 can predict outcomes, including recovery times, efficacy based on clinical end points, likelihood of success, predicted mortality, predicted related follow-up surgeries, or the like. In some embodiments, the server 106 can continuously or periodically analyze patient data (including patient data obtained during the patient stay) to determine near real-time or real-time risk scores, mortality prediction, etc.

In some embodiments, the server 106 includes one or more modules for performing one or more steps of the patient-specific treatment planning methods described herein. For example, in the depicted embodiment, the server 106 includes a data analysis module 116, a treatment planning module 118, a disease progression module 120, and an intervention timing module 121. In alternative embodiments, one or more of these modules may be combined with each other, or may be omitted. Thus, although certain operations are described herein with respect to a particular module or modules, this is not intended to be limiting, and such operations can be performed by a different module or modules in alternative embodiments.

The data analysis module 116 is configured with one or more algorithms for identifying a subset of reference data from the database 110 that is likely to be useful in developing a patient-specific treatment plan. For example, the data analysis module 116 can compare patient-specific data (e.g., the patient data set 108 received from the client computing device 102) to the reference data from the database 110 (e.g., the reference patient data sets) to identify similar data (e.g., one or more similar patient data sets in the reference patient data sets). The comparison can be based on one or more parameters, such as age, gender, BMI, lumbar lordosis, pelvic incidence, and/or treatment levels. The parameter(s) can be used to calculate a similarity score for each reference patient. The similarity score can represent a statistical correlation between the patient data set 108 and the reference patient data set. Accordingly, similar patients can be identified based on whether the similarity score is above, below, or at a specified threshold value. For example, as described in greater detail below, the comparison can be performed by assigning values to each parameter and determining the aggregate difference between the subject patient and each reference patient. Reference patients whose aggregate difference is below a threshold can be considered to be similar patients.

The data analysis module 116 can further be configured with one or more algorithms to select a subset of the reference patient data sets, e.g., based on similarity to the patient data set 108 and/or treatment outcome of the corresponding reference patient. For example, the data analysis module 116 can identify one or more similar patient data sets in the reference patient data sets, and then select a subset of the similar patient data sets based on whether the similar patient data set includes data indicative of a favorable or desired treatment outcome. The outcome data can include data representing one or more outcome parameters, such as corrected anatomical metrics, presence of fusion, HRQL, activity level, complications, recovery times, efficacy, mortality, or follow-up surgeries. As described in further detail below, in some embodiments, the data analysis module 116 calculates an outcome score by assigning values to each outcome parameter. A patient can be considered to have a favorable outcome if the outcome score is above, below, or at a specified threshold value.

In some embodiments, the data analysis module 116 selects a subset of the reference patient data sets based at least in part on user input (e.g., from a clinician, surgeon, physician, healthcare provider). For example, the user input can be used in identifying similar patient data sets. In some embodiments, weighting of similarity and/or outcome parameters can be selected by a healthcare provider or physician to adjust the similarity and/or outcome score based on clinician input. In further embodiments, the healthcare provider or physician can select the set of similarity and/or outcome parameters (or define new similarity and/or outcome parameters) used to generate the similarity and/or outcome score, respectively.

In some embodiments, the data analysis module 116 includes one or more algorithms used to select a set or subset of the reference patient data sets based on criteria other than patient parameters. For example, the one or more algorithms can be used to select the subset based on healthcare provider parameters (e.g., based on healthcare provider ranking/scores such as hospital/physician expertise, number of procedures performed, hospital ranking, etc.) and/or healthcare resource parameters (e.g., diagnostic equipment, facilities, surgical equipment such as surgical robots), or other non-patient related information that can be used to predict outcomes and risk profiles for procedures for the present healthcare provider. For example, reference patient data sets with images captured from similar diagnostic equipment can be aggregated to reduce or limit irregularities due to variation between diagnostic equipment. Additionally, patient-specific treatment plans can be developed for a particular health-care provider using data from similar healthcare providers (e.g., healthcare providers with traditionally similar outcomes, physician expertise, surgical teams, etc.). In some embodiments, reference healthcare provider data sets, hospital data sets, physician data sets, surgical team data sets, post-treatment data set, and other data sets can be utilized. By way of example, a patient-specific surgical plan to perform a battlefield surgery can be based on reference patient data from similar battlefield surgeries and/or datasets associated with battlefield surgeries. In another example, the patient-specific surgical plan can be generated based on available robotic surgical systems. The reference patient data sets can be selected based on patients that have been operated on using comparable robotic surgical systems under similar conditions (e.g., size and capabilities of surgical teams, hospital resources, etc.).

The treatment planning module 118 is configured with one or more algorithms to generate at least one surgical plan (e.g., pre-operative plans, intra-operative plans, post-operative plans etc.) based on the output from the data analysis module 116. In some embodiments, the treatment planning module 118 is configured to develop and/or implement at least one predictive model for generating the patient-specific treatment plan, also known as a “prescriptive model.” The predictive model(s) can be developed using clinical knowledge, statistics, machine learning, AI, neural networks, or the like. In some embodiments, the output from the data analysis module 116 is analyzed (e.g., using statistics, machine learning, neural networks, AI) to identify correlations between data sets, patient parameters, healthcare provider parameters, healthcare resource parameters, treatment procedures, medical device designs, and/or treatment outcomes. These correlations can be used to develop at least one predictive model that predicts the likelihood that a surgical plan will produce a favorable outcome for the particular patient. The predictive model(s) can be validated, e.g., by inputting data into the model(s) and comparing the output of the model to the expected output.

In some embodiments, the treatment planning module 118 is configured to generate the surgical plan based on previous treatment data from reference patients. For example, the treatment planning module 118 can receive a selected subset of reference patient data sets and/or similar patient data sets from the data analysis module 116, and determine or identify treatment data from the selected subset. The treatment data can include, for example, treatment procedure data (e.g., surgical procedure or intervention data) and/or medical device design data (e.g. implant design data) that are associated with favorable or desired treatment outcomes for the corresponding patient. The treatment planning module 118 can analyze the treatment procedure data and/or medical device design data to determine an optimal treatment protocol for the patient to be treated. For example, the treatment procedures and/or medical device designs can be assigned values and aggregated to produce a treatment score. The patient-specific surgical plan can be determined by selecting surgical plan(s) based on the score (e.g., higher or highest score; lower or lowest score; score that is above, below, or at a specified threshold value). The personalized patient-specific surgical plan can be based on, at least in part, the patient-specific technologies or patient-specific selected technology.

Alternatively or in combination, the treatment planning module 118 can generate the surgical plan based on correlations between data sets. For example, the treatment planning module 118 can correlate treatment procedure data and/or medical device design data from similar patients with favorable outcomes (e.g., as identified by the data analysis module 116). Correlation analysis can include transforming correlation coefficient values to values or scores. The values/scores can be aggregated, filtered, or otherwise analyzed to determine one or more statistical significances. These correlations can be used to determine surgical procedure(s) and/or medical device design(s) that are optimal or likely to produce a favorable outcome for the patient to be treated.

Alternatively or in combination, the treatment planning module 118 can generate the surgical plan using one or more AI techniques. AI techniques can be used to develop computing systems capable of simulating aspects of human intelligence, e.g., learning, reasoning, planning, problem solving, decision making, etc. AI techniques can include, but are not limited to, case-based reasoning, rule-based systems, artificial neural networks, decision trees, support vector machines, regression analysis, Bayesian networks (e.g., naïve Bayes classifiers), genetic algorithms, cellular automata, fuzzy logic systems, multi-agent systems, swarm intelligence, data mining, machine learning (e.g., supervised learning, unsupervised learning, reinforcement learning), and hybrid systems.

In some embodiments, the treatment planning module 118 generates the surgical plan using one or more trained machine learning models. Various types of machine learning models, algorithms, and techniques are suitable for use with the present technology. In some embodiments, the machine learning model is initially trained on a training data set, which is a set of examples used to fit the parameters (e.g., weights of connections between “neurons” in artificial neural networks) of the model. For example, the training data set can include any of the reference data stored in database 110, such as a plurality of reference patient data sets or a selected subset thereof (e.g., a plurality of similar patient data sets).

In some embodiments, the machine learning model (e.g., a neural network or a naïve Bayes classifier) may be trained on the training data set using a supervised learning method (e.g., gradient descent or stochastic gradient descent). The training dataset can include pairs of generated “input vectors” with the associated corresponding “answer vector” (commonly denoted as the target). The current model is run with the training data set and produces a result, which is then compared with the target, for each input vector in the training data set. Based on the result of the comparison and the specific learning algorithm being used, the parameters of the model are adjusted. The model fitting can include both variable selection and parameter estimation. The fitted model can be used to predict the responses for the observations in a second data set called the validation data set. The validation data set can provide an unbiased evaluation of a model fit on the training data set while tuning the model parameters. Validation data sets can be used for regularization by early stopping, e.g., by stopping training when the error on the validation data set increases, as this may be a sign of overfitting to the training data set. In some embodiments, the error of the validation data set error can fluctuate during training, such that ad-hoc rules may be used to decide when overfitting has truly begun. Finally, a test data set can be used to provide an unbiased evaluation of a final model fit on the training data set.

To generate a surgical plan, the patient data set 108 can be input into the trained machine learning model(s). Additional data, such as the selected subset of reference patient data sets and/or similar patient data sets, and/or treatment data from the selected subset, can also be input into the trained machine learning model(s). The trained machine learning model(s) can then calculate whether various candidate treatment procedures and/or medical device designs are likely to produce a favorable outcome for the patient. Based on these calculations, the trained machine learning model(s) can select at least one surgical plan for the patient. In some embodiments, the trained machine learning model(s) can determine candidate procedures (or candidate surgical plans), analyze the candidate procedures, select the candidate surgical plans or portions thereof, score plans, and/or generate surgical plans for the patient. Each surgical plan can be scored (e.g., scored based on favorable outcome, likelihood of outcome, etc.) and ranked according to the score. The trained machine learning model(s) can determine a set of surgical plans that meet selection criteria for plan review by a user. The selection criteria can be based on, for example, regulatory requirements, reimbursement criteria, healthcare/provider expertise, available surgical equipment, manufacturing capabilities, elimination criteria, combinations thereof, or the like. A user can input one or more selection criteria to control the types and/or features of the surgical plans for comparison. In embodiments where multiple trained machine learning models are used, the models can be run sequentially or concurrently to compare outcomes and can be periodically updated using training data sets. The treatment planning module 118 can use one or more of the machine learning models based the model's predicted accuracy score.

The patient-specific surgical plan generated by the treatment planning module 118 can include at least one patient-specific surgical procedure (e.g., a surgical procedure or intervention) and/or at least one patient-specific medical device (e.g., an implant or implant delivery instrument). A patient-specific surgical plan can include an entire surgical procedure or portions thereof. Additionally, one or more patient-specific medical devices can be specifically selected or designed for the corresponding surgical procedure, thus allowing for the various components of the patient-specific technology to be used in combination to treat the patient.

In some embodiments, the patient-specific surgical procedure includes an orthopedic surgery procedure, such as spinal surgery, hip surgery, knee surgery, jaw surgery, hand surgery, shoulder surgery, elbow surgery, total joint reconstruction (arthroplasty), skull reconstruction, foot surgery, or ankle surgery. Spinal surgery can include spinal fusion surgery, such as posterior lumbar interbody fusion (PLIF), anterior lumbar interbody fusion (ALIF), transverse or transforaminal lumbar interbody fusion (TLIF), lateral lumbar interbody fusion (LLIF), direct lateral lumbar interbody fusion (DLIF), or extreme lateral lumbar interbody fusion (XLIF). In some embodiments, the patient-specific treatment procedure includes descriptions of and/or instructions for performing one or more aspects of a patient-specific surgical procedure. For example, the patient-specific surgical procedure can include one or more of a surgical approach, a corrective maneuver, a bony resection, or implant placement.

In some embodiments, the patient-specific medical device design includes a design for an orthopedic implant and/or a design for an instrument for delivering an orthopedic implant. Examples of such implants include, but are not limited to, screws (e.g., bone screws, spinal screws, pedicle screws, facet screws), interbody implant devices (e.g., intervertebral implants), cages, plates, rods, disks, fusion devices, spacers, rods, expandable devices, stents, brackets, ties, scaffolds, fixation device, anchors, nuts, bolts, rivets, connectors, tethers, fasteners, joint replacements, hip implants, or the like. Examples of instruments include, but are not limited to, screw guides, cannulas, ports, catheters, insertion tools, or the like.

A patient-specific medical device design can include data representing one or more of physical properties (e.g., size, shape, volume, material, mass, weight), mechanical properties (e.g., stiffness, strength, modulus, hardness), and/or biological properties (e.g., osteo-integration, cellular adhesion, anti-bacterial properties, anti-viral properties) of a corresponding medical device. For example, a design for an orthopedic implant can include implant shape, size, material, and/or effective stiffness (e.g., lattice density, number of struts, location of struts, etc.). In some embodiments, the generated patient-specific medical device design is a design for an entire device. Alternatively, the generated design can be for one or more components of a device, rather than the entire device.

In some embodiments, the design is for one or more patient-specific device components that can be used with standard, off-the-shelf components. For example, in a spinal surgery, a pedicle screw kit can include both standard components and patient-specific customized components. In some embodiments, the generated design is for a patient-specific medical device that can be used with a standard, off-the-shelf delivery instrument. For example, the implants (e.g., screws, screw holders, rods) can be designed and manufactured for the patient, while the instruments for delivering the implants can be standard instruments. This approach allows the components that are implanted to be designed and manufactured based on the patient's anatomy and/or surgeon's preferences to enhance treatment. The patient-specific devices described herein are expected to improve delivery into the patient's body (e.g., in conjunction with planned surgical procedures or steps), placement at the treatment site, and/or interaction with the patient's anatomy.

In embodiments where the patient-specific surgical plan includes a specific surgical procedure to implant a medical device, the treatment planning module 118 can also store various types of implant surgery information, such as implant parameters (e.g., types, dimensions), availability of implants, aspects of a pre-operative plan (e.g., initial implant configuration, detection and measurement of the patient's anatomy, etc.), FDA requirements for implants (e.g., specific implant parameters and/or characteristics for compliance with FDA regulations), or the like. In some embodiments, the treatment planning module 118 can convert the implant surgery information into formats useable for machine-learning based models and algorithms. For example, the implant surgery information can be tagged with particular identifiers for formulas or can be converted into numerical representations suitable for supplying to the trained machine learning model(s). The treatment planning module 118 can also store information regarding the patient's anatomy, such as two- or three-dimensional images or models of the anatomy, and/or information regarding the biology, geometry, and/or mechanical properties of the anatomy. The anatomy information can be used to inform implant design and/or placement.

The disease progression module 120 can be used to analyze, predict, and/or model disease progression for a particular patient. As described in detail below, the disease progression module 120 can estimate the rate of disease progression for the patient under a variety of different circumstances, including (a) if no surgical intervention occurs, and (b) if one or more surgical plans (e.g., surgical procedures identified by the treatment planning module 118) are performed. The disease progression module 120 can therefore include an algorithm, machine learning model, or other software analytical tool for predicting disease progression in a particular patient.

In some embodiments, the disease progression module 120 includes a machine learning model or other software module that can be trained based off a plurality of reference patient data sets that includes, in addition to the patient data described above, disease progression metrics for each of the reference patients. The progression metrics can include measurements for disease metrics over a period of time. Suitable metrics may include spinopelvic parameters (e.g., lumbar lordosis, pelvic tilt, sagittal vertical axis (SVA), cobb angel, coronal offset, etc.), disability scores, functional ability scores, flexibility scores, VAS pain scores, or the like. The progression of the metrics for each reference patient can be correlated to other patient information for the specific reference patient (e.g., age, sex, height, weight, activity level, diet, etc.). The disease metrics can include values over a period of time. For example, the reference patient data may include values of disease metrics on a daily, weekly, monthly, bi-monthly, yearly, or other basis. By measuring the metrics over a period of time, changes in the values of the metrics can be tracked as an estimate of disease progression and correlated to other patient data.

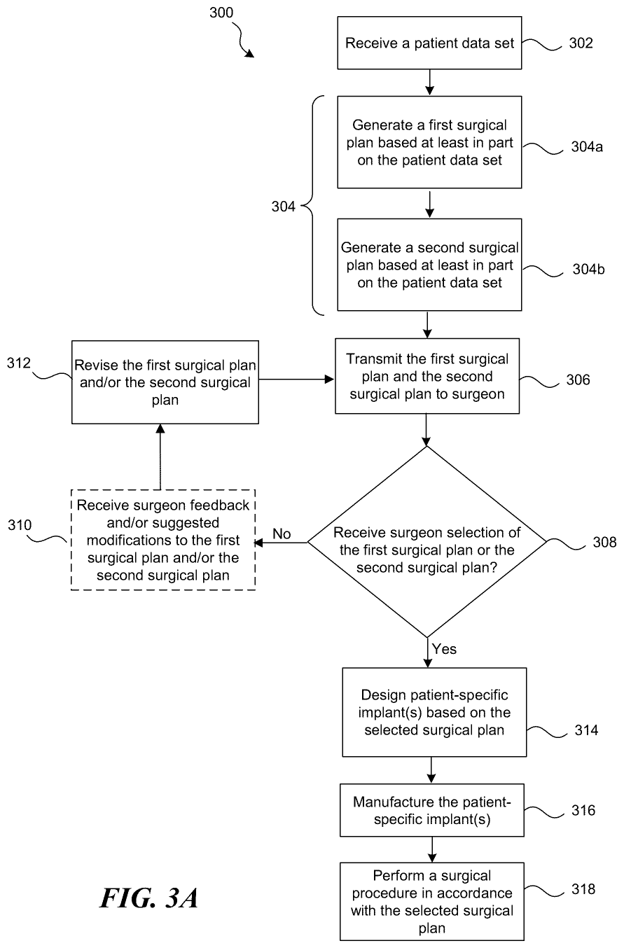

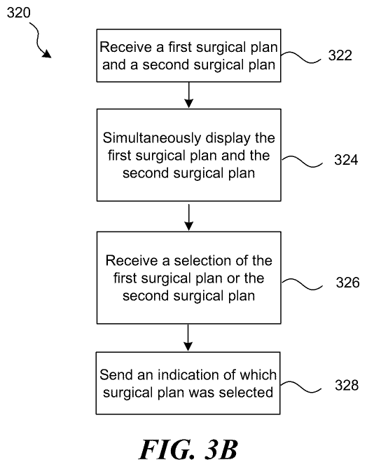

In some embodiments, the disease progression module 120 can therefore estimate the rate of disease progression for a particular patient. The progression may be estimated by providing estimated changes in one or more disease metrics over a period of time (e.g., X % increase in a disease metric per year). The rate can be constant (e.g., 5% increase in pelvic tilt per year) or variable (e.g., 5% increase in pelvic tilt for a first year, 10% increase in pelvic tilt for a second year, etc.). In some embodiments, the estimated rate of progression can be transmitted to a surgeon or other healthcare provider as part of a surgical plan, as described in greater detail below with respect to FIGS. 3A-5.

As a non-limiting example, a particular patient who is a fifty-five-year-old male may have a SVA value of 6 mm. The disease progression module 120 can analyze patient reference data sets to identify disease progression for individual reference patients having one or more similarities with the particular patient (e.g., individual patients of the reference patients who have an SVA value of about 6 mm and are approximately the same age, weight, height, and/or sex of the patient). Based on this analysis, the disease progression module 120 can predict the rate of disease progression if no surgical intervention occurs (e.g., the patient's VAS pain scores may increase 5%, 10%, or 15% annually if no surgical intervention occurs, the SVA value may continue to increase by 5% annually if no surgical intervention occurs, etc.).

The surgical treatment plans and/or associated patient-specific implants described herein can also be at least partially based on the estimated rates of disease progression, enabling the modeling of different outcomes over a desired period of times. Additionally, the models/simulations can account for any number of additional diseases or conditions to predict the patient's overall health, mobility, or the like. These additional diseases or conditions can, in combination with other patient health factors (e.g., height, weight, age, activity level, etc.) be used to generate a patient health score reflecting the overall health of the patient. The patient health score can be displayed for surgeon review and/or incorporated into the estimation of disease progression. Accordingly, the present technology can generate one or more virtual simulations of the predicted disease progression to demonstrate how the patient's anatomy is predicted to change over time. Physician input can be used to generate or modify the virtual simulation(s). The present technology can generate one or more post-treatment virtual simulations based on the received physician input for review by the healthcare provider, patient, etc.

In some embodiments, the present technology can also predict, model, and/or simulate disease progression based on one or more potential surgical plans. For example, the disease progression module 120 may simulate what a patient's anatomy and/or spinal metrics may be 1, 2, 5, or 10 years post-surgery for several different surgical plans. The simulations may also incorporate non-surgical factors, such as patient age, height, weight, sex, activity level, other health conditions, or the like, as previously described. The system and/or a surgeon can use the disease progression to aid in selecting which surgical plan provides the best long-term efficacy, as described below with respect to FIGS. 3A-5. These simulations can also be used to determine patient-specific corrections that compensate for the projected diseases progression.

Accordingly, in some embodiments, multiple disease progression models (e.g., two, three, four, five, six, or more) are simulated to provide disease progression data for several different surgical plans. For example, the disease progression module can generate models that predict post-surgical disease progression for each of three different surgical plans. A surgeon or other healthcare provider can review the disease progression models and, based on the review, select which of the three surgical plans is likely to provide the patient with the best long-term outcome.

Based off of the modeled disease progression, the systems and methods described herein can also (i) identify a recommended time for surgical intervention, and/or (ii) identify a recommended type of surgical procedure for the patient. In some embodiments, the present technology therefore includes an intervention timing module 121 that includes an algorithm, machine learning model, or other software analytical tool for determining the optimal time for surgical intervention in a particular patient. This can be done, for example, by analyzing patient reference data that includes (i) pre-operative disease progression metrics for individual reference patients, (ii) disease metrics at the time of surgical intervention for individual reference patients, (iii) post-operative disease progression metrics for individual reference patients, and/or (iv) scored surgical outcomes for individual reference patients. The intervention timing module 121 can compare the disease metrics for a particular patient to the reference patient data sets to determine, for similar patients, the point of disease progression at which surgical intervention produced the most favorable outcomes.

As a non-limiting example, the reference patient data sets may include data associated with reference patients' sagittal vertical axis. The data can include (i) sagittal vertical axis values for individual patients over a period of time before surgical intervention (e.g., how fast and to what degree the sagittal vertical axis value changed), (ii) sagittal vertical axis of the individual patients at the time of surgical intervention, (iii) the change in sagittal vertical axis after surgical intervention, and (iv) the degree to which the surgical intervention was successful (e.g., based on pain, quality of life, or other factors). Based on the foregoing data, the intervention timing module 121 can, based on a particular patient's sagittal vertical axis value, identify at which point surgical intervention will have the highest likelihood of producing the most favorable outcome. Of course, the foregoing metric is provided by way of example only, and the intervention timing module 121 can incorporate other metrics (e.g., lumbar lordosis, pelvic tilt, sagittal vertical axis, cobb angel, coronal offset, disability scores, functional ability scores, flexibility scores, VAS pain scores) instead of or in combination with sagittal vertical axis to predict the time at which surgical intervention has the highest probability of providing a favorable outcome for the particular patient.

The intervention timing module 121 may also incorporate one or more mathematical rules based on value thresholds for various disease metrics. For example, the intervention timing module 121 may indicate surgical intervention is necessary if one or more disease metrics exceed a predetermined threshold or meet some other criteria. Representative thresholds that indicate surgical intervention may be necessary include SVA values greater than 7 mm, a mismatch between lumbar lordosis and pelvic incidence greater than 10 degrees, a cobb angle of greater than 10 degrees, and/or a combination of cobb angle and LL/PI mismatch greater than 20 degrees. Of course, other threshold values and metrics can be used; the foregoing are provided as examples only. In some embodiments, the foregoing rules can be tailored to specific patient populations (e.g., for males over 50 years of age, an SVA value greater than 7 mm indicates the need for surgical intervention). If a particular patient does not exceed the thresholds indicating surgical intervention is recommended, the intervention timing module 121 may provide an estimate for when the patient's metrics will exceed one or more thresholds, thereby providing the patient with an estimate of when surgical intervention may become recommended.

In some embodiments, the treatment planning module 118 identifies one or more types of surgical procedures for the patient based at least in part on the disease progression of the patient determined using the disease progression module 120 and/or the intervention timing module 121. The treatment planning module 118 may also incorporate one or more mathematical rules for identifying surgical procedures. As a non-limiting example, if a LL/PI mismatch is between 10 and 20 degrees, the treatment planning module 118 may recommend an anterior fusion surgery, but if the LL/PI mismatch is greater than 20 degrees, the treatment planning module may recommend both anterior and posterior fusion surgery. As another non-limiting example, if a SVA value is between 7 mm and 15 mm, the treatment planning module may recommend posterior fusion surgery, but if the SVA is above 15 mm, the treatment planning module may recommend both posterior fusion surgery and anterior fusion surgery. Of course, other rules can be used; the foregoing are provided as examples only.

Without being bound by theory, incorporating disease progression modeling into the patient-specific surgical plans described herein may even further increase the effectiveness of the procedures and/or provide a surgeon more data by which to evaluate various surgical plans. For example, in many cases it may be disadvantageous to operate after a patient's disease progresses to an irreversible or unstable state. However, it may also be disadvantageous to operate too early, such as before the patient's disease is causing symptoms and/or if the patient's disease may not progress further. The disease progression module 120 and/or the intervention timing module 121 can therefore help identify the window of time during which surgical intervention in a particular patient has the highest probability of providing a favorable outcome for the patient.

The surgical plan(s) generated by the treatment planning module 118 can be transmitted via the communication network 104 to the client computing device 102 for output to a user (e.g., clinician, surgeon, healthcare provider, patient). In some embodiments, the client computing device 102 includes or is operably coupled to a display 122 for outputting the treatment plan(s). The display 122 can include a graphical user interface (GUI) for visually depicting various aspects of the surgical plan(s). For example, the display 122 can show various aspects of a surgical procedure to be performed on the patient, such as the surgical approach, treatment levels, corrective maneuvers, tissue resection, and/or implant placement. To facilitate visualization, the surgical plan can include a virtual model of the surgical procedure that can be displayed via the display 122. The display 122 may also display additional aspects of the surgical plan, such as predicted post-operative patient metrics, predicted disease progression metrics associated with the identified surgical procedure, etc. As another example, the display 122 can show a design for a medical device to be implanted in the patient in accordance with the transmitted surgical plan, such as a two- or three-dimensional model of the device design. The display 122 can also show patient information, such as two- or three-dimensional images or models of the patient's anatomy where the surgical procedure is to be performed and/or where the device is to be implanted. An example of the display 122 depicting a surgical plan is provided below at FIGS. 4A-5. The client computing device 102 can further include one or more user input devices (not shown) allowing the user to modify, select, approve, and/or reject the displayed treatment plan(s).

In some embodiments, the medical device design(s) generated by the treatment planning module 118 can be transmitted from the client computing device 102 and/or server 106 to a manufacturing system 124 for manufacturing a corresponding medical device. The manufacturing system 124 can be located on site or off site. On-site manufacturing can reduce the number of sessions with a patient and/or the time to be able to perform the surgery whereas off-site manufacturing can be useful make the complex devices. Off-site manufacturing facilities can have specialized manufacturing equipment. In some embodiments, more complicated device components can be manufactured off site, while simpler device components can be manufactured on site.

Various types of manufacturing systems are suitable for use in accordance with the embodiments herein. For example, the manufacturing system 124 can be configured for additive manufacturing, such as three-dimensional (3D) printing, stereolithography (SLA), digital light processing (DLP), fused deposition modeling (FDM), selective laser sintering (SLS), selective laser melting (SLM), selective heat sintering (SHM), electronic beam melting (EBM), laminated object manufacturing (LOM), powder bed printing (PP), thermoplastic printing, direct material deposition (DMD), inkjet photo resin printing, or like technologies, or combination thereof. Alternatively or in combination, the manufacturing system 124 can be configured for subtractive (traditional) manufacturing, such as CNC machining, electrical discharge machining (EDM), grinding, laser cutting, water jet machining, manual machining (e.g., milling, lathe/turning), or like technologies, or combinations thereof. The manufacturing system 124 can manufacture one or more patient-specific medical devices based on fabrication instructions or data (e.g., CAD data, 3D data, digital blueprints, stereolithography data, or other data suitable for the various manufacturing technologies described herein). Different components of the system 100 can generate at least a portion of the manufacturing data used by the manufacturing system 124. The manufacturing data can include, without limitation, fabrication instructions (e.g., programs executable by additive manufacturing equipment, subtractive manufacturing equipment, etc.), 3D data, CAD data (e.g., CAD files), CAM data (e.g., CAM files), path data (e.g., print head paths, tool paths, etc.), material data, tolerance data, surface finish data (e.g., surface roughness data), regulatory data (e.g., FDA requirements, reimbursement data, etc.), or the like. The manufacturing system 124 can analyze the manufacturability of the implant design based on the received manufacturing data. The implant design can be finalized by altering geometries, surfaces, etc. and then generating manufacturing instructions. In some embodiments, the server 106 generates at least a portion of the manufacturing data, which is transmitted to the manufacturing system 124.

The manufacturing system 124 can generate CAM data, print data (e.g., powder bed print data, thermoplastic print data, photo resin data, etc.), or the like and can include additive manufacturing equipment, subtractive manufacturing equipment, thermal processing equipment, or the like. The additive manufacturing equipment can be 3D printers, stereolithography devices, digital light processing devices, fused deposition modeling devices, selective laser sintering devices, selective laser melting devices, electronic beam melting devices, laminated object manufacturing devices, powder bed printers, thermoplastic printers, direct material deposition devices, or inkjet photo resin printers, or like technologies. The subtractive manufacturing equipment can be CNC machines, electrical discharge machines, grinders, laser cutters, water jet machines, manual machines (e.g., milling machines, lathes, etc.), or like technologies. Both additive and subtractive techniques can be used to produce implants with complex geometries, surface finishes, material properties, etc. The generated fabrication instructions can be configured to cause the manufacturing system 124 to manufacture the patient-specific orthopedic implant that matches or is therapeutically the same as the patient-specific design. In some embodiments, the patient-specific medical device can include features, materials, and designs shared across designs to simplify manufacturing. For example, deployable patient-specific medical devices for different patients can have similar internal deployment mechanisms but have different deployed configurations. In some embodiments, the components of the patient-specific medical devices are selected from a set of available pre-fabricated components and the selected pre-fabricated components can be modified based on the fabrication instructions or data.

The surgical plans described herein can be performed by a surgeon, a surgical robot, or a combination thereof, thus allowing for treatment flexibility. In some embodiments, the surgical procedure can be performed entirely by a surgeon, entirely by a surgical robot, or a combination thereof. For example, one step of a surgical procedure can be manually performed by a surgeon and another step of the procedure can be performed by a surgical robot. In some embodiments the treatment planning module 118 generates control instructions configured to cause a surgical robot (e.g., robotic surgery systems, navigation systems, etc.) to partially or fully perform a surgical procedure. The control instructions can be transmitted to the robotic apparatus by the client computing device 102 and/or the server 106.

Following the treatment of the patient in accordance with the surgical plan, treatment progress can be monitored over one or more time periods to update the data analysis module 116, treatment planning module 118, disease progression module 120, and/or intervention timing module 121. Post-treatment data can be added to the reference data stored in the database 110. The post-treatment data can be used to train machine learning models for developing patient-specific treatment plans, patient-specific medical devices, or combinations thereof.

It shall be appreciated that the components of the system 100 can be configured in many different ways. For example, in alternative embodiments, the database 110, the data analysis module 116, the treatment planning module 118, the disease progression module 120, and/or the intervention timing module 121 can be components of the client computing device 102, rather than the server 106. As another example, the database 110, the data analysis module 116, the treatment planning module 118, the disease progression module 120, and/or the intervention timing module 121 can be located across a plurality of different servers, computing systems, or other types of cloud-computing resources, rather than at a single server 106 or client computing device 102.

Additionally, in some embodiments, the system 100 can be operational with numerous other computing system environments or configurations. Examples of computing systems, environments, and/or configurations that may be suitable for use with the technology include, but are not limited to, personal computers, server computers, handheld or laptop devices, cellular telephones, wearable electronics, tablet devices, multiprocessor systems, microprocessor-based systems, programmable consumer electronics, network PCs, minicomputers, mainframe computers, distributed computing environments that include any of the above systems or devices, or the like.

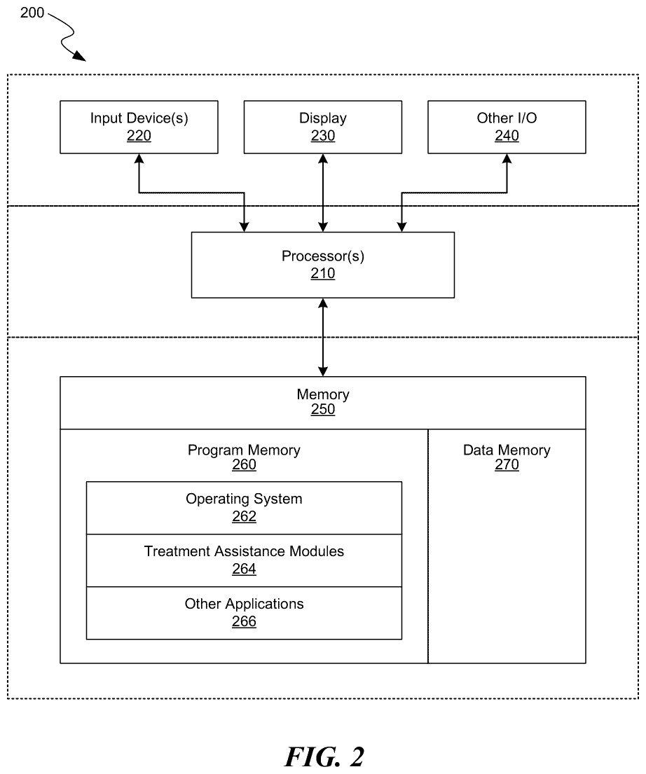

FIG. 2 illustrates a computing device 200 suitable for use in connection with the system 100 of FIG. 1, according to an embodiment. The computing device 200 can be incorporated in various components of the system 100 of FIG. 1, such as the client computing device 102 or the server 106. The computing device 200 includes one or more processors 210 (e.g., CPU(s), GPU(s), H P U (s), etc.). The processor(s) 210 can be a single processing unit or multiple processing units in a device or distributed across multiple devices. The processor(s) 210 can be coupled to other hardware devices, for example, with the use of a bus, such as a PCI bus or SCSI bus. The processor(s) 210 can be configured to execute one more computer-readable program instructions, such as program instructions to carry out of any of the methods described herein.

The computing device 200 can include one or more input devices 220 that provide input to the processor(s) 210, e.g., to notify it of actions from a user of the device 200. The actions can be mediated by a hardware controller that interprets the signals received from the input device and communicates the information to the processor(s) 210 using a communication protocol. Input device(s) 220 can include, for example, a mouse, a keyboard, a touchscreen, an infrared sensor, a touchpad, a wearable input device, a camera- or image-based input device, a microphone, or other user input devices.

The computing device 200 can include a display 230 used to display various types of output, such as text, models, virtual procedures, surgical plans, implants, graphics, and/or images (e.g., images with voxels indicating radiodensity units or Hounsfield units representing the density of the tissue at a location). In some embodiments, the display 230 provides graphical and textual visual feedback to a user. The processor(s) 210 can communicate with the display 230 via a hardware controller for devices. In some embodiments, the display 230 includes the input device(s) 220 as part of the display 230, such as when the input device(s) 220 include a touchscreen or is equipped with an eye direction monitoring system. In alternative embodiments, the display 230 is separate from the input device(s) 220. Examples of display devices include an LCD display screen, an LED display screen, a projected, holographic, or augmented reality display (e.g., a heads-up display device or a head-mounted device), and so on.

Optionally, other I/O devices 240 can also be coupled to the processor(s) 210, such as a network card, video card, audio card, USB, firewire or other external device, camera, printer, speakers, CD-ROM drive, DVD drive, disk drive, or Blu-Ray device. Other I/O devices 240 can also include input ports for information from directly connected medical equipment such as imaging apparatuses, including MRI machines, X-Ray machines, CT machines, etc. Other I/O devices 240 can further include input ports for receiving data from these types of machine from other sources, such as across a network or from previously captured data, for example, stored in a database.

In some embodiments, the computing device 200 also includes a communication device (not shown) capable of communicating wirelessly or wire-based with a network node. The communication device can communicate with another device or a server through a network using, for example, TCP/IP protocols. The computing device 200 can utilize the communication device to distribute operations across multiple network devices, including imaging equipment, manufacturing equipment, etc.

The computing device 200 can include memory 250, which can be in a single device or distributed across multiple devices. Memory 250 includes one or more of various hardware devices for volatile and non-volatile storage, and can include both read-only and writable memory. For example, a memory can comprise random access memory (RAM), various caches, CPU registers, read-only memory (ROM), and writable non-volatile memory, such as flash memory, hard drives, floppy disks, CDs, DVDs, magnetic storage devices, tape drives, device buffers, and so forth. A memory is not a propagating signal divorced from underlying hardware; a memory is thus non-transitory. In some embodiments, the memory 250 is a non-transitory computer-readable storage medium that stores, for example, programs, software, data, or the like. In some embodiments, memory 250 can include program memory 260 that stores programs and software, such as an operating system 262, one or more treatment assistance modules 264, and other application programs 266. The treatment assistance module(s) 264 can include one or more modules configured to perform the various methods described herein (e.g., the data analysis module 116 and/or treatment planning module 118 described with respect to FIG. 1). Memory 250 can also include data memory 270 that can include, e.g., reference data, configuration data, settings, user options or preferences, etc., which can be provided to the program memory 260 or any other element of the computing device 200.

### B. Select Methods of Modeling and Designing Multiple Patient-Specific Surgical Plans

The present technology includes systems and methods for generating a plurality of patient-specific surgical plans that can be analyzed and compared to select an optimal surgical plan. As described below, the patient-specific surgical plans can include, among other things, a surgical procedure and a predicted post-operative outcome for the patient if the surgical procedure were to be performed. The predicted post-operative outcomes for each surgical plan can be compared to identify the best surgical plan for the particular patient.

FIG. 3A is a flow diagram illustrating a method 300 for providing patient-specific medical care, according to an embodiment of the present technology. Some or all of the method 300 can be performed by various computing systems or software modules, including, for example, the computing systems described above with respect to FIGS. 1 and 2.

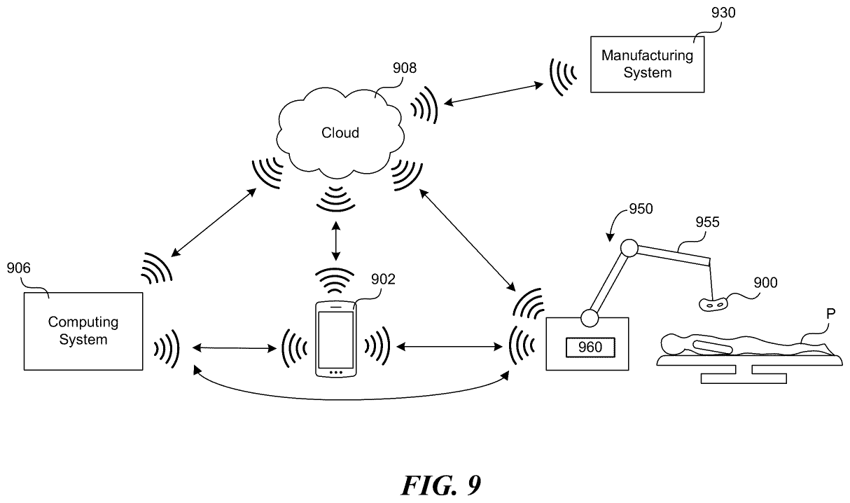

The method 300 can begin in block 302 by receiving a patient data set for a particular patient in need of medical treatment. The patient data set can include data representative of the patient's condition, anatomy, pathology, symptoms, medical history, preferences, and/or any other information or parameters relevant to the patient. For example, the patient data set can include surgical intervention data, treatment outcome data, progress data (e.g., surgeon notes), patient feedback (e.g., feedback acquired using quality of life questionnaires, surveys), clinical data, patient information (e.g., demographics, sex, age, height, weight, type of pathology, occupation, activity level, tissue information, health rating, comorbidities, health related quality of life (HRQL)), vital signs, diagnostic results, medication information, allergies, diagnostic equipment information (e.g., manufacturer, model number, specifications, user-selected settings/configurations, etc.) or the like. The patient data set can also include image data, such as camera images, Magnetic Resonance Imaging (MRI) images, ultrasound images, Computerized Aided Tomography (CAT) scan images, Positron Emission Tomography (PET) images, X-Ray images, and the like. In some embodiments, the patient data set includes data representing one or more of patient identification number (ID), age, gender, body mass index (BMI), lumbar lordosis, Cobb angle(s), pelvic incidence, disc height, segment flexibility, bone quality, rotational displacement, and/or treatment level of the spine. The patient data set can be received at a server, computing device, or other computing system. For example, in some embodiments the patient data set can be received by the server 106 shown in FIG. 1 or the computing system 906 described below with respect to FIG. 9. In some embodiments, the computing system that receives the patient data set in block 302 also stores one or more software modules (e.g., the data analysis module 116, the treatment planning module 118, the disease progression module 120, and/or the intervention timing module 121, shown in FIG. 1, or additional software modules for performing various operations of the method 300).

In some embodiments, the received patient data set can include disease metrics such as lumbar lordosis, Cobb angles, coronal parameters (e.g., coronal balance, global coronal balance, coronal pelvic tilt, etc.), sagittal parameters (e.g., pelvic incidence, sacral slope, thoracic kyphosis, etc.) and/or pelvic parameters. The disease metrics can include micro-measurements (e.g., metrics associated with specific or individual segments of the patient's spine) and/or macro-measurements (e.g., metrics associated with multiple segments of the patient's spine). In some embodiments, the disease metrics are not included in the patient data set, and the method 300 includes determining (e.g., automatically determining) one or more of the disease metrics based on the patient image data, as described below. In some embodiments, the received patient data can include functional mobility test scores (e.g., step test, six-meter walk test, sit-to-stand test, timed up and go test, etc.). The received patient data set can include additional subjective test scores that reflect aspects of the patient condition, such as pain tests (e.g., Visual Analog Scale (VAS) pain scores, Low Back Pain Rating scale scores, etc.), disability tests (e.g., Oswestry Disability Index scores, Quebec back pain disability test scores, etc.), quality of life tests (e.g., Quality of Life Scale scores), etc.

The method 300 can continue in block 304 by generating two or more surgical plans based at least in part on the patient data set received in block 302. As described in detail below, the two or more surgical plans can each include a target location or region of interest for surgical intervention and one or more surgical procedures or interventions to be performed at the region of interest. Each surgical plan can also include predicted post-operative data associated with performing the surgical procedure at the target location. For example, each surgical plan may include a predicted or target post-operative anatomical configuration shown as a two or three dimensional virtual model. In some embodiments, the surgical plans include additional predicted post-operative analytics, such as predicted disease progression, predicted patient satisfaction, predicted patient mobility, predicted patient pain, predicted patient quality of life, etc.

The surgical plans generated at block 304 can be generated simultaneously or sequentially using the same or different techniques. For example, the method 300 can include, at block 304a, generating a first surgical plan based at least in part on the patient data set received in block 304a. In some embodiments, the operation of generating the first surgical plan includes identifying a specific target location to be involved in the surgical procedure. For example, in the context of spinal surgery, generating the first surgical plan may include identifying one or more vertebral levels for surgical intervention. In some embodiments, the vertebral level is a cervical vertebral level (e.g., C1-C5), a thoracic vertebral level (e.g., T1-T12), a lumbar vertebral level (e.g., L1-L5), and/or the sacrum. In some embodiments, the identified target location includes a specific range of vertebral levels to be involved in a surgery (e.g., L1-L3, L3-L5, L4-T12, C1-C3, etc.). The identified target location may include two, three, four, five, or more vertebral levels. Of course, the foregoing target locations are provided by way of example only, and the present technology is not limited to the anatomical locations listed above. Indeed, in some embodiments the target location may include anatomical structures other than the spine, such as the hip, knee, ankle, shoulder, elbow, wrist, hand, the jaw, the skull, or other anatomical locations, as described throughout this Detailed Description.

The target location can be identified by reviewing image data of the patient. In some embodiments, a computing system (e.g., the computing system 106 of FIG. 1) and/or one or more software modules (e.g., the treatment planning module 118 of FIG. 1) can review and analyze patient image data and automatically identify the target location. In such embodiments, a trained machine learning program or other software-based program can analyze patient image data, extract measurements from the patient image data, compare the extracted measurements to reference data (e.g., predetermined thresholds or ranges associated with “healthy” patients normalized for age, sex, gender, etc.), and identify anatomical regions that are candidates for surgical correction. Alternatively or additionally, the target location can be identified and/or confirmed through other suitable means, such as via a technician or healthcare provider reviewing image data and identifying anatomical deformities.

As provided above, in some embodiments the operation of generating the first surgical plan also includes identifying a surgical procedure for the patient. In embodiments in which the surgical plan includes identifying a target location, the surgical procedure can be associated with the target location. In the context of spinal surgery, representative surgical procedures include spinal fusion, artificial disc replacement, vertebroplasty, kyphoplasty, spinal laminectomy/decompression, discectomy, facetectomy, foraminotomy, or other spine surgery procedures. Examples of spinal fusion surgery include posterior lumbar interbody fusion (PLIF), anterior lumbar interbody fusion (ALIF), transverse or transforaminal lumbar interbody fusion (TLIF), lateral lumbar interbody fusion (LLIF), direct lateral lumbar interbody fusion (DLIF), or extreme lateral lumbar interbody fusion (XLIF). The foregoing are provided by way of example only, and the present technology can include identifying any type of spinal or other surgical procedures in block 304a.

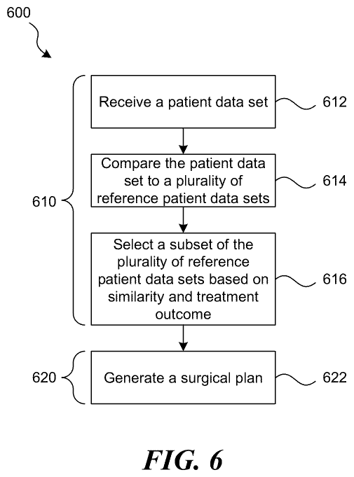

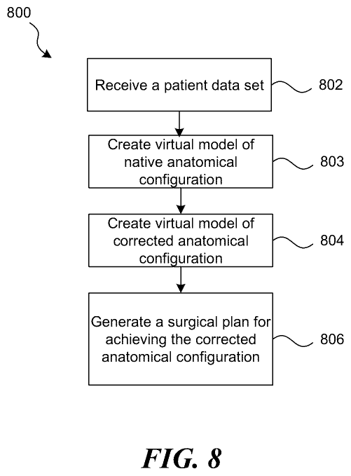

The surgical procedure associated with the first surgical plan can be identified using any of the methods and systems described herein. For example, in some embodiments the computing system 106 of FIG. 1 and/or the associated software modules (e.g., the treatment planning module 118) can identify one or more surgical procedures based on, for example, user input, the received or extracted patient data, and/or the identified target location(s). For example, if the computing system 106 determines that a patient is suffering from disc degeneration from L3-L5, the computing system 106 may recommend a PLIF procedure to fuse L2-T12. Alternatively, the computing system 106 may recommend an artificial disc replacement at L3-L4 and L4-L5 to correct the degeneration while preserving motion. The surgical procedure can be identified using other methods and systems as well. For example, additional methods for identifying surgical procedures for inclusion in surgical plans are described with respect to FIGS. 6 and 8.

In some embodiments, step 304 can include reviewing and/or analyzing multiple types of surgical procedures and/or surgical steps to identify the surgical procedure for inclusion within the first surgical plan. Types of surgical procedures and/or surgical steps can be selected for inclusion with the first surgical plan (or eliminated from inclusion with the first surgical plan) based on, for example, user input, insurance coverage of the procedure or step, healthcare provider parameters (e.g., based on healthcare provider ranking/scores such as hospital/physician expertise, number of similar procedures performed, hospital ranking for procedure, etc.), healthcare resource parameters (e.g., diagnostic equipment, facilities, surgical equipment such as surgical robots), and/or other non-patient related information (e.g., information that can be used to score, predict outcomes and risk profiles for procedures for the present healthcare provider, and/or rank procedures).

In some embodiments, the operation of generating the first surgical plan includes identifying or designing a corrected anatomical configuration for the patient (the corrected anatomical configuration can also be referred to herein as the “planned configuration,” “optimized geometry,” “post-operative anatomical configuration,” or “target outcome”). The corrected anatomical configuration can reflect the desired and/or predicted anatomy of the patient if the surgical plan were performed. In some embodiments, generating the first surgical plan includes generating one or more virtual models (two-dimensional models, three-dimensional models, etc.) showing the corrected anatomical configuration. The virtual model may include some or all of the patient's anatomy within the target location (e.g., any combination of tissue types including, but not limited to, bony structures, cartilage, soft tissue, vascular tissue, nervous tissue, etc.). In some embodiments, the corrected anatomical configuration is identified/determined before the surgical procedure and/or target location. That is, a computing system or user can model a preferred anatomical outcome, and, based on the desired anatomical outcome, identify a surgical procedure and target location that will achieve the desired anatomical outcome once performed.

In some embodiments, generating the first surgical plan includes generating one or more patient metrics associated with the corrected anatomical configuration. In the context of spinal surgery, patient metrics may include, for example, coronal parameters, sagittal parameters, pelvic parameters, Cobb angles, shoulder tilt, iliolumbar angles, coronal balance, lordosis angles, intervertebral space height, or other similar spinal parameters. Similar as described above, the patient metrics can be determined before identifying a surgical procedure and/or target location for surgical intervention. That is, a computing system or user can use the patient metrics to identify a surgical procedure and target location that will achieve the patient metrics once performed.

In some embodiments, the corrected anatomical configuration is designed by one or more software modules, computing systems, or the like. Representative methods and techniques for designing corrected anatomical configurations are described in greater detail with respect to FIG. 8. Likewise, in some embodiments the patient metrics associated with the corrected anatomical configuration can be automatically or semi-automatically extracted from a model of the corrected anatomical configuration, as described below with respect to FIG. 8.

The first surgical plan can include additional features. In some embodiments, for example, the first surgical plan can include predicted disease progression, predicted patient satisfaction, predicted patient mobility, predicted patient pain, predicted patient quality of life, or the like. For example, the first surgical plan may include estimates of disease progression if the patient were to undergo the identified surgical procedure at the identified target location. That is, the surgical plan can include virtual models (e.g., two-dimensional or three-dimensional virtual models) of patient anatomy at various intervals post-operation. For example, the surgical plan may include a predictive model of patient anatomy at one or more of 6 months post-op, 1 year post-op, 2 years post-op, 3 years post-op, 4 year post-op, 5 years post-op, 6 years post-op, 7 years post-op, 8 years post-op, 9 years post-op, and/or 10 years post-op. The disease progression model may also include predicted patient metrics (e.g., any of the patient metrics described herein, including coronal parameters, sagittal parameters, pelvic parameters, Cobb angles, shoulder tilt, iliolumbar angles, coronal balance, lordosis angles, intervertebral space height, or other similar spinal parameters) at any of the various post-operative intervals identified above, in addition to or in lieu of including the virtual model of predicted patient anatomy.

Once generated, the surgical plan can be digitally displayed as a surgical report on one or more display screens for ease of review, editing, annotation, the like. For example, the computing system may store the surgical plan as computer executable instructions that, when executed by a processor, causes the computing system to display the surgical plan for review. An example of a surgical plan displayed for review is described below with respect to FIGS. 4A-5.

The method 300 can also include, at block 304b, generating a second surgical plan based at least in part on the patient data set. Similar to the first surgical plan, the second surgical plan can one or more target locations or regions of interest for surgical intervention, one or more surgical procedures or interventions, a target post-operative anatomical configuration, and/or predicted post-operative patient data. The second surgical plan can include any of target locations for surgical intervention, surgical procedures, and target post-operative anatomical configurations described above with respect to the first surgical plan. However, the second surgical plan is at least partially different than the first surgical plan. For example, the second surgical plan may include the same target location but a different surgical procedure (e.g., if the first surgical plan recommended L2-L5 fusion, the second surgical plan may recommend artificial disc replacement from L2-L5).

In some embodiments, the second surgical plan includes a different target location or region of interest for surgical intervention than the first surgical plan. In such embodiments, the target location may at least partially overlap with the target location of the first surgical plan. For example, if the first surgical plan recommends surgical intervention at a vertebral range of L3-L5, the second surgical plan may recommend surgical intervention at a vertebral range of L2-L4, or just at L4. In other embodiments, the target location for the second surgical plan does not overlap with the target location for the first surgical plan. For example, if the first surgical plan recommends surgical intervention at a vertebral range of L3-L5, the second surgical plan may recommend surgical intervention at S1-L2. In other embodiments, the target location for the second surgical plan can be the same as the target location for the first surgical plan.

In some embodiments, the second surgical plan includes a different surgical procedure than the first surgical plan. For example, if the first surgical plan recommends a spinal fusion surgery, the second surgical plan may recommend a non-fusion surgery, such as an artificial disc replacement. In some embodiments, the first surgical plan and the second surgical plan may recommend the same general surgical approach (e.g., spinal fusion), but may recommend different techniques (e.g., PLIF vs. ALIF). In other embodiments, the surgical procedure in the second surgical plan can be the same as the surgical procedure in the first surgical plan.

In some embodiments, the second surgical plan includes a different target post-operative anatomical configuration than the first surgical plan. For example, in the context of spinal surgery, the second target surgical outcome may include a post-operative anatomical configuration having different coronal parameters, sagittal parameters, pelvic parameters, Cobb angles, shoulder tilt, iliolumbar angles, coronal balance, lordosis angles, intervertebral space height, or other similar spinal parameters than those associated with the first surgical plan.

Similar to the first surgical plan, the second surgical plan can include additional features beyond those described above. In some embodiments, for example, the second surgical plan can include predicted disease progression, predicted patient satisfaction, predicted patient mobility, predicted patient pain, predicted patient quality of life, or the like associated with the identified surgical procedure and/or the identified target location.

Although only two surgical plans are shown as being generated at block 304 (i.e., the first surgical plan in block 304a and the second surgical plan in block 304b), the method 300 can include generating any number of surgical plans using the same or generally similar process as described above with respect to blocks 304a and 304b. That is, the method 300 can generate one, two, three, four, five, six, seven, eight, or more unique surgical plans. Each of the surgical plans can provide a different potential surgical intervention for the patient to be treated. Accordingly, although the method is described with respect to generating a first surgical plan and a second surgical plan, one skilled in the art will appreciate that the method 300 can include generating more than two surgical plans.

In some embodiments, the operation of generating the first surgical plan at block 304a and generating the second surgical plan at block 304b includes generating a plurality of candidate surgical plans (or subsets of surgical plans such as surgical procedures), and then selecting the first surgical plan and the second surgical plan from within the plurality of candidate surgical plans. For example, in some embodiments a computing system can automatically identify a plurality (e.g., two, three, four, five, six, seven, eight, nine, ten, or more) of surgical plans (or subsets of surgical plans such as surgical procedures) based on the patient-data set and/or one or more user-inputted criteria. The identified candidate surgical plans may be ranked and/or scored based on various factors, including predicted patient outcomes, user-review, etc. The highest ranked identified candidate surgical plans (e.g., based on predicted patient outcomes) can be selected as the first surgical plan and the second surgical plan. In some embodiments, certain ranked surgical plans may not be selected as the first surgical plan or the second surgical plan based on user review and/or failure to meet various user criteria. For example, if a particular surgical plan is identified as requiring a surgical procedure that the physician is unfamiliar with, the particular surgical plan may not be selected, and the method can instead include selecting the next best surgical plan as the first surgical plan. Accordingly, in some implementations, physician-specific scoring is used to score candidate procedures/surgical plans before selecting the first surgical plan and/or the second surgical plan. For example, procedures with scores meeting a threshold score (e.g., threshold post-operative metrics score, physician inputted threshold score, threshold outcome score, etc.) can be identified for user review. The system can therefore compare advantages and disadvantages of candidate procedures with respect to each other before selecting the first surgical plan and/or second surgical plan.

Accordingly, in some embodiments, to select surgical plans for comparisons, the system can determine a score for each candidate surgical plan. The system can rank the surgical plans based on the determined scores. The system can select a set of surgical plans for comparison based on the ranking. For example, the system can select a predetermined number of the highest ranked surgical plans for review by the user. The user can input or modify scoring protocols to provide for flexibility. In some embodiments, threshold scoring can be used to select the candidate surgical plans for comparison. For example, the system can determine a score for each of the surgical plans. The system can select a set of surgical plans (e.g., the first surgical plan and the second surgical plan), each with scores above a threshold score. The threshold score can be determined using a machine learning algorithm trained using prior patient data with threshold scores. In some embodiments, a user can input a threshold score. For example, a physician can input a threshold score based on the physician's diagnosis of the patient. The system can compare the surgical plans in the set for user review. The comparison can include a report with side-by-side metrics, images of virtual models, and other information discussed in connection with FIGS. 4A-5.

In some embodiments, the system receives user input before generating the candidate surgical plans. For example, a user may provide user input for specific surgical procedures. The system can then analyze the patient data to identify candidate surgical procedures based at least in part on the user inputted specific surgical procedures. For each of the candidate surgical procedures, the system can generate a respective surgical plan with one or more virtual post-operative anatomical models for the patient. Other user input that can be used to generate the candidate surgical plans can include, without limitation, at least one of type of procedure (e.g., posterior lumbar interbody fusion, disc replacement, interbody fusion and disc replacement, rod-based fusion, expandable fusion cages, non-expandable fusion cages, etc.), one or more levels for treatment, insurance or payment information for the patient, or elimination criteria (e.g., candidate procedure or surgical step elimination criteria). The insurance information can be acquired by the system to determine whether the procedure will be covered by the patient's insurance. The system can also generate a comparison of two or more candidate surgical plans for user review. In some embodiments, the user can approve, modify, and/or reject the candidate surgical plans.

The system can select candidate procedures and/or surgical plans based on the user input using a machine learning model trained using prior patent data sets that meet the candidate procedure criteria to identify candidate procedures for generating surgical plans. The prior patient data sets can be for patients treated by, for example, the same physician or a group of physicians. Other types of patient data sets can be used.

In some embodiments, the system can provide viewing of the candidate surgical plans for modification, elimination, and/or approval by a user before selecting the first surgical plan and the second surgical plan. The system can then generate the first surgical plan and/or the second surgical plan based on user input received after generating the candidate surgical plans. In some embodiments, the user input can include one or more elimination criteria. For example, if a patient has a pre-existing condition that prevents interbody fusion procedures at more than a predetermined number of levels of the spine, the user can input the number of levels set as the maximum number of levels for treatment. The system can eliminate candidate procedures/surgical plans that include implants at more than the predetermined number of levels. The candidate procedure elimination criteria can be per spinal segment, per spine level, etc.

In some embodiments, the user input for sorting the candidate surgical plans can include specifying types of surgical procedures for inclusion/exclusion, including open procedures, minimally-invasive procedures, manually performed procedures, robotic-assisted procedures, or the like. This allows the user to select candidate procedures/surgical plans suitable for being performed using available surgical suites, surgical equipment, hospitals, or the like. For example, if a robotic surgical system is available, the user can select robotically-performed or assisted surgical procedures to generate robotic surgical plans. If robotic surgical systems are not available, the surgeon can select manually performed surgical procedures. In some embodiments, the user input is provided before the candidate surgical plans are generated, as set forth above. For example, the user input can specify a type of surgical procedure, and the system can generate candidate surgical plans that only include the specified surgical procedure. In some embodiments, the system can also identify surgical suites available for the candidate surgical procedure and acquire surgical equipment information for those surgical suites. Based on the acquired information, the system can generate surgical plans suitable for performing surgeries at an available surgical suite.

Once the first surgical plan and the second surgical plan are generated in block 304, the method 300 can continue in block 306 by transmitting the first surgical plan and the second surgical plan to a surgeon. In some embodiments, the first and second surgical plans are transmitted as first and second surgical plan reports, examples of which are shown in FIGS. 4A-5. In some embodiments, the same computing system used in blocks 302 and 304 can transmit the first and second surgical plans to a computing device for surgeon review (e.g., the client computing device 102 described in FIG. 1 or the computing device 902 described below with respect to FIG. 9). This can include directly transmitting the first and second surgical plans to the computing device or uploading the first and second surgical plans to a cloud or other storage system for subsequent downloading.

The surgeon can review and compare the first and second surgical plans and, in block 308, select to proceed with either the first surgical plan or the second surgical plan. For example, the first surgical plan and the second surgical plan can be displayed to the surgeon side by side to enable the surgeon to review the first surgical plan and the second surgical plan and determine which surgical plan is preferred for the particular patient. In some embodiments, this includes a side-by-side comparison of (a) the first surgical plan's target locations, surgical procedure, target/predicted post-operative anatomical configuration, and predicted post-operative patient metrics with (b) the second surgical plan's target locations, surgical procedure, target/predicted post-operative anatomical configuration, and predicted post-operative patient metrics. In some embodiments, this also includes a side-by-side comparison of a model of the first surgical plan's target post-operative anatomical configuration and the second surgical plan's target post-operative anatomical configuration. In this way, a surgeon can easily review and compare which of the first and second surgical plans is preferred for the patient. Additional details regarding displaying multiple surgical plans for surgeon review are described below with respect to the method 320. An example of a side-by-side comparison of multiple surgical plans is shown in FIG. 5 and described below.

In some embodiments, the surgeon may not approve or select either the first surgical plan or the second surgical plan reviewed in block 308. In such embodiments, the surgeon can optionally provide feedback and/or suggested modifications to the surgical plan (e.g., by adjusting the virtual model or changing one or more aspects about the plan). Accordingly, the method 300 can optionally include receiving (e.g., via the computing system) the surgeon feedback and/or suggested modifications in block 310. This may include, for example, modifying target locations for surgical intervention, surgical procedures, and/or target post-operative anatomical configuration. If surgeon feedback and/or suggested modifications are received in block 310, the method 300 can continue in block 312 by revising (e.g., automatically revising via the computing system) the first surgical plan and/or the second surgical plan based at least in part on the surgeon feedback and/or suggested modifications received in block 310. In some embodiments, the surgeon does not provide feedback and/or suggested modifications if they reject the surgical plan. In such embodiments, block 310 can be omitted, and the method 300 can continue in block 312 by revising (e.g., automatically revising via the computing system) the first and second surgical plans by selecting new and/or additional reference patient data sets and/or generating third and fourth surgical plans. The revised and/or new surgical plans can then be transmitted to the surgeon for review. The operations in blocks 306, 308, 310, and 312 can be repeated as many times as necessary until the surgeon selects and approves a particular surgical plan.

Once surgeon approval of a surgical plan is received in block 308, the method 300 can continue in block 314 by designing (e.g., via the same computing system that performed blocks 302-308) one or more patient-specific implants based on the selected surgical plan. For example, the patient-specific implant can be designed based on the target location and surgical procedure included in the selected surgical plan. The patient-specific implant(s) can also be specifically designed such that, when implanted in the particular patient at the target location using the identified surgical procedure, it directs the patient's anatomy to occupy the target post-operative anatomical configuration (e.g., transforming the patient's anatomy from the patient's native anatomical configuration to the corrected anatomical configuration). The patient-specific implant can be designed such that, when implanted, it causes the patient's anatomy to occupy the corrected anatomical configuration for the expected service life of the implant (e.g., 5 years or more, 10 years or more, 20 years or more, 50 years or more, etc.). In some embodiments, the patient-specific implant is designed solely based on the virtual model of the corrected anatomical configuration and/or without reference to pre-operative patient images.

The patient-specific implant can be any of the implants described herein or in any patent references incorporated by reference herein. For example, the patient-specific implant can include one or more of screws (e.g., bone screws, spinal screws, pedicle screws, facet screws), interbody implant devices (e.g., intervertebral implants), cages, plates, rods, discs, fusion devices, spacers, rods, expandable devices, stents, brackets, ties, scaffolds, fixation device, anchors, nuts, bolts, rivets, connectors, tethers, fasteners, joint replacements (e.g., artificial discs), hip implants, or the like. A patient-specific implant design can include data representing one or more of physical properties (e.g., size, shape, volume, material, mass, weight), mechanical properties (e.g., stiffness, strength, modulus, hardness), and/or biological properties (e.g., osteo-integration, cellular adhesion, anti-bacterial properties, anti-viral properties) of the implant. For example, a design for an orthopedic implant can include implant shape, size, material, and/or effective stiffness (e.g., lattice density, number of struts, location of struts, etc.). An example of a patient-specific implant designed via the method 300 is described below with respect to FIGS. 12A and 12B. Additional examples of patient-specific implants that can be designed at block 314 are described in U.S. application Ser. Nos. 16/048,167, 16/242,877, 16/207,116, 16/352,699, 16/383,215, 16/569,494, 16/699,447, 16/735,222, 16/987,113, 16/990,810, 17/085,564, 17/100,396, 17/342,329, 17/518,524, 17/531,417, 17/835,777, 17/851,487, 17/867,621, and 17/842,242, each of which is incorporated by reference herein in its entirety.

In some embodiments, designing the implant in block 316 can optionally include generating fabrication instructions for manufacturing the implant. For example, the computing system may generate computer-executable fabrication instructions that that, when executed by a manufacturing system, cause the manufacturing system to manufacture the implant.

In some embodiments, the patient-specific implant is designed in block 316 only after the surgeon has selected a surgical plan. Accordingly, in some embodiments, the implant design is neither transmitted to the surgeon with the surgical plan in block 308, nor manufactured before receiving surgeon approval of the surgical plan. Without being bound by theory, waiting to design the patient-specific implant until after the surgeon approves the surgical plan may increase the efficiency of the method 300 and/or reduce the resources necessary to perform the method 300. In other embodiments, one or more patient-specific implants can be designed and included in the surgical plans transmitted to the surgeon at block 306. Accordingly, in some embodiments the operation in block 314 can be included within the block 304.

The method 300 can continue in block 316 by manufacturing the patient-specific implant. The implant can be manufactured using additive manufacturing techniques, such as 3D printing, stereolithography, digital light processing, fused deposition modeling, selective laser sintering, selective laser melting, electronic beam melting, laminated object manufacturing, powder bed printing, thermoplastic printing, direct material deposition, or inkjet photo resin printing, or like technologies, or combination thereof. Alternatively or additionally, the implant can be manufactured using subtractive manufacturing techniques, such as CNC machining, electrical discharge machining (EDM), grinding, laser cutting, water jet machining, manual machining (e.g., milling, lathe/turning), or like technologies, or combinations thereof. The implant may be manufactured by any suitable manufacturing system (e.g., the manufacturing system 124 shown in FIG. 1 or the manufacturing system 930 described below with respect to FIG. 9). In some embodiments, the implant is manufactured by the manufacturing system executing the computer-readable fabrication instructions generated by the computing system in block 316.

Once the implant is manufactured in block 316, the method 300 can continue in block 318 by performing the selected surgical plan and implanting the patient-specific implant into the patient. Aspects of the surgical plan, such as some or all of the surgical procedure, can be performed manually, by a robotic surgical platform (e.g., a surgical robot), or a combination thereof. In embodiments in which the surgical procedure is performed at least in part by a robotic surgical platform, the surgical plan can include computer-readable control instructions configured to cause the surgical robot to perform, at least partly, the patient-specific surgical procedure. Additional details regarding a robotic surgical platform are described below with respect to FIG. 9.

The method 300 can be implemented and performed in various ways. In some embodiments, the operations in blocks 302-314 can be performed by a computing system associated with a first entity, block 316 can be performed by a manufacturing system associated with a second entity, and block 318 can be performed by a surgical provider, surgeon, and/or robotic surgical platform associated with a third entity. Any of the foregoing blocks may also be implemented as computer-readable instructions stored in memory and executable by one or more processors of the associated computing system(s).

FIG. 3B is a flowchart of a method 320 of providing patient specific medical care, accordingly to embodiments of the present technology. More specifically, the method 320 is directed to comparing and selecting between a plurality of surgical plans, such as the first and second surgical plans described with respect to the method 300 of FIG. 3A. Accordingly, the method 320 can be a computer-implemented method performed via a client computing device, such as a computer, tablet, phone, or the like (e.g., the computing device 102 of FIG. 1).

The method 320 can begin in block 322 by receiving a first surgical plan and a second surgical plan. In some embodiments, the first surgical plan and the second surgical plan are received at the client computing device by downloading the first surgical plan and the second surgical plan from a server or cloud-based storage platform. In other embodiments, the first surgical plan and the second surgical plan are directly received at the client computing device from another computing system (e.g., the system 106) via Wi-Fi, cellular, Bluetooth, NFC, or another communication network.

The method 324 can continue in block 324 by simultaneously displaying the first surgical plan and the second surgical plan. This can be done via a display screen of the client computing device (e.g., the tablet or computer screen), such as the display 122 of FIG. 1. The first surgical plan and the second surgical plan can be displayed “side-by-side” such that at least some aspects of the first surgical plan and the second surgical plan are displayed at the same time. For example, block 324 may include simultaneously displaying, via the display screen, the first surgical plan's target location for surgical intervention and the second surgical plan's target location for surgical intervention. Additionally or alternatively, block 324 may include simultaneously displaying, via the display screen, the first surgical plan's surgical procedure and the second surgical plan's surgical procedure. Additionally or alternatively, block 324 may include simultaneously displaying, via the display screen, the first surgical plan's target post-operative anatomical configuration and the seconds surgical plan's target post-operative anatomical configuration. This may include displaying a model of the target post-operative anatomical configuration and/or metrics associated with the target post-operative anatomical configuration. In some embodiments, the display screen is interactive such that a user (e.g., a surgeon) can toggle between different aspects of the surgical plans, zoom in or out (e.g., on the models of target post-operative anatomical configuration), annotate the plans, provide feedback on the plans, etc. An example of a side-by-side display of multiple surgical plans is provided in FIG. 5 below.

The method 320 can continue in block 326 by receiving a selection of the first surgical plan or the second surgical plan. This can include, for example, receiving a selection from a user (e.g., a surgeon) indicating whether the user prefers to proceed with the first surgical plan or the second surgical plan. The method 320 can continue in block 328 by sending an indication of which surgical plan was selected. In some embodiments, this includes sending an indication to the computing system that originally transmitted the first and second surgical plans to the client computing device and/or uploaded the first and seconds surgical plans to the server.

As described above, the surgical plans described herein can include a target surgical location, a surgical procedure, and predicted post-operative patient metrics, among other data. FIG. 4A illustrates a first portion of a representative surgical plan 400 displayed via the display 122 according to an embodiment of the present technology. In some embodiments, the surgical plan is generated as described above in block 304 of the method 300.

As illustrated, the surgical plan 400 includes a patient name (or, alternatively, an anonymized patient identifier), a region of interest (e.g., target surgical location), a surgical procedure, and an implant type and number associated with the surgical procedure. The surgical plan 400 further includes patient images 402 showing pre-operative patient anatomy. Although shown as radiographic images, in other embodiments other image types may be included (e.g., MRI), or the pre-operative images may be omitted. The surgical plan 400 further includes pre-operative patient metrics 404 and post-operative patient metrics 406. As set forth above, the post-operative patient metrics are predicted patient metrics for the patient if the surgical plan is selected and performed by the surgeon. The surgical plan 400 further includes a first three-dimensional representation or virtual model 410 (“first virtual model 410”) showing a pre-operative (e.g., native) anatomical configuration of the patient and a second three-dimensional representation or virtual model 420 (“second virtual model 420”) showing a predicted post-operative anatomical configuration of the patient. In the illustrated embodiment, the first virtual model 410 and the second virtual model 420 are three-dimensional models showing the patients lumbar spinal region, although in other embodiments the first virtual model 410 and/or the second virtual model 420 can be two-dimensional and include more or less patient anatomy. In some embodiments, the first virtual model 410 is omitted entirely, and only the second virtual model 420 showing post-operative anatomical configuration is provided. Optionally, the second virtual model 420 may also include one or more virtual implants 422 implanted at the target locations.

FIG. 4B illustrates a second portion of the representative surgical plan 400 displayed via the display screen according to an embodiment of the present technology. In some embodiments, the second portion of the surgical plan 400 shown in FIG. 4B is continuous with the first portion shown in FIG. 4A, and can be accessed simply by scrolling along the display 122. In other embodiments, the second portion can represent a second “page” of the surgical plan 400, and can be accessed by scrolling along the display 122 and/or toggling a button identified as “next page” (not shown).

The second portion of the surgical plan 400 includes disease progression predictions associated with the surgical plan 400. For example, the post-operative disease progression predictions include estimated post-operative patient metrics 408a for 1 year post-operation, estimated post-operative patient metrics 408b for 3 years post-operation, and estimated post-operative patient metrics 408c for 5 years post-operation. The post-operative disease progression predictions also includes a first virtual model 424a showing predicted patient anatomy 1 year post-operation, a second virtual model 424b showing predicted patient anatomy 3 years post-operation, and a third virtual model 424c showing predicted patient anatomy 5 years post-operation. Without being bound by theory, including patient disease progression predictions associated with the surgical intervention is expected to provide the surgeon a more long-term outlook on patient outcomes if the surgical plan is performed, as compared to embodiments in which only immediate post-operative metrics and models are provided.

The surgical plan 400 is provided by way of example only. One skilled in the art will appreciate from the disclosure herein that many iterations and variations of surgical plans can be generated using the systems and methods of the present technology. Accordingly, the present technology is not limited to the specific representation of the surgical plan provided in FIGS. 4A and 4B, unless clearly expressed otherwise. Moreover, although depicted on the display 122, the surgical plan 400 can be displayed via other suitable display screens, and/or stored via computer-executable instructions on a computing device, server, or other cloud-based storage systems. The surgical plan 400 can also include additional information not shown in FIGS. 4A and 4B, such as future surgical procedures, physician information (e.g., physician-specific expertise, physician-specific prior surgical outcomes, etc.), regulatory information (e.g., regulatory requirements, regulatory-based reimbursements information, etc.), insurance or payment information (e.g., coverage for some or all of the surgical plan), reimbursement criteria, healthcare/provider expertise, available surgical equipment, manufacturing capabilities, combinations thereof, or the like.

FIG. 5 illustrates an interactive surgical planner 501 with a side-by-side comparison of the surgical plan 400 shown in FIG. 4A and a surgical plan 500 according to an embodiment of the present technology. In some embodiments, the surgical plan 400 can be the first surgical plan generated at block 304a of the method 300 of FIG. 3A, and the surgical plan 500 can be the second surgical plan generated at block 304b of the method 300 of FIG. 3A. Thus, the surgical plan 400 can be referred to as the first surgical plan 400, and the surgical plan 500 can be referred to as the second surgical plan 500.

As shown, the first surgical plan 400 and the second surgical plan 500 can be displayed (e.g., via the display 122) next to each other such that a surgeon or other user can simultaneously view and compare aspects of the both the first surgical plan 400 and the second surgical plan 500. In some embodiments, similar features of the first surgical plan 400 appear next to similar features of the second surgical plan 500 for ease of comparison. For example, in the illustrated embodiment, the predicted post-operative patient metrics 406 of the first surgical plan 400 appear next to predicted post-operative patient metrics 506 of the second surgical plan 500. Likewise, the virtual model 420 including the virtual implants 422 of the target post-operative anatomical configuration associated with the first surgical plan 400 is next to a virtual model 520 including virtual implants 522 of a target post-operative anatomical configuration associated with the second surgical plan 500. Although only certain features of the first surgical plan 400 and the second surgical plan 500 are shown in FIG. 5, one skilled in the art will appreciate that the side-by-side comparison of the first surgical plan 400 and the second surgical plan 500 can include any surgical plan features described herein, including those discussed with respect to FIGS. 3A-4B.

In some embodiments, a user can select a comparison button (not shown) to compare various aspects of the first surgical plan 400 and the second surgical plan 500. For example, the system can compare disease progression the first surgical plan 400 and the second surgical plan 500 for a user defined period of time (e.g., 1 year, 2 years, 3 years, etc.). As another example, the system can compare predicted mobility for the patient for both the first surgical plan 400 and the second surgical plan 500. For example, the first surgical plan 400 and the second surgical plan 500 may each include predicted mobility metrics (e.g., predicted range of motion, overall mobility scores, etc.) based on whether the first surgical plan 400 or the second surgical plan 500 is implemented. Such data may be useful in embodiments in which the predicted post-operative anatomical configuration is the same or generally similar between the first surgical plan 400 and the second surgical plan 500, but the procedure and implant type are different (e.g., fusion vs. disc replacement). In some implementations, the system can compare the first surgical plan 400 and the second surgical plan 500 based on the physician's expertise, prior outcomes, etc. For example, if the physician has more favorable outcomes with interbody fusion procedures than disc replacement procedures for similar prior patients, the system can recommend the first surgical plan that includes an interbody device with physician-specific interbody fusion analytics compared to physician-specific disc replacement analytics. This allows a user to select plans based on user-selected comparisons. The system can also display whether a plan will be covered by a patient's insurance, including criteria for imbursement.

In some embodiments, a user can modify and/or eliminate from consideration one or more of the displayed surgical plans. For example, a user may input elimination criteria that can eliminate and/or modify the displayed surgical plans, as described previously. Elimination criteria can include types of surgical steps or procedures. For example, a physician can input certain types of procedures (e.g., a fusion procedure, disc replacement, rod fusion procedure, interbody fusion procedure, etc.) to be eliminated or excluded surgical plans. The physician can select the elimination criteria based on his/her evaluation of the patient.

In some embodiments, a user can select features of the interactive surgical planner 501 for generating a new surgical plan based on one or more modifications to the displayed interactive surgical plan. This allows a user to provide level-by-level modification, selection, and approval of a surgical plan. In some implementations, for example, a physician can select one or more of the virtual implants 422 of the first surgical plan 400 that provide desired spinal level outcomes. The user can then select one or more implants 522 of the second surgical plan 500 that provide spinal level outcomes. The system can then generate a new surgical plan based on a combination of the selected implants 422, 522. That is, the system can merge aspects of the first surgical plan 400 and the second surgical plan 500 based on user input to create a third surgical plan. The third surgical plan can include modified implants 422, 522 designed to provide a comprehensive overall desired anatomical configuration. In some embodiments, the system can indicate whether one or more implants from a surgical plan can be combined with one or more implants or another surgical plan. This informs the user of potential design options.

The interactive surgical planner 501 can be used to modify the first surgical plan 400 and/or the second surgical plan 500 by, for example, receiving at least one post-operative metric from a user. For example, the displayed metrics can be replaced by new metrics using drop down menus, fillable boxes, etc. The corresponding virtual anatomical model used to generate the plan can be modified to meet the post-operative metric(s). The modified virtual anatomical model can be measured to obtain a modified set of predicted post-operative spinal metrics. This allows a physician to change metrics and then evaluate predicted post-operative spine configuration, reciprocal changes along the spine, etc. For example, images of the virtual models (e.g., virtual models 420, 520) and associated set of predicted post-operative spinal metrics can be updated in near-real time or real-time.

The virtual models can be linked to an account of the patient to enable coordinated modifications using software, including anatomical modeling software, computer-aided design (CAD) software, etc. User inputted metrics can be automatically feed into the software to modify the virtual model, analyze the virtual model, etc. The virtual model can have repositionable anatomical elements a first model data for measuring the first post-operative spinal metrics of the at least one virtual anatomical model in a first post-operative configuration for a first surgical procedure and second model data for determining second post-operative spinal metrics the at least one virtual anatomical model in a first post-operative configuration associated with a second surgical procedure. The model data (e.g., first model data and/or second model data) can include, for example, parametric data, surface rendering data, metric measuring algorithms, and/or image data from images of the patient. The interactive surgical planner 501 can display the generated data, provide user input features (e.g., boxes, fillable fields, drop down menus, etc.), and dynamic viewing of models. In some embodiments, the user can use a touchscreen to rotate, pan, crop, reposition anatomical elements, or otherwise manipulate the virtual models. This allows the user to view anatomy from different perspectives in near real-time or real-time. The user can adjust the patient metrics and then view how those adjustments are predicted to affect other anatomical features. In pre-operative settings, the physician and patient can both view the proposed surgical plan, metrics, and anatomical models to prepare for the surgical procedure. In intra-operative settings, the physician can compare intra-operative anatomical positions to planned anatomical positions. In post-operative settings, the physician can compare post-operative anatomical positions to planned post-operative position to monitor treatment outcomes.

The interactive surgical planner 501 can be used to manage data transformations, available data sources, rules for managing conflicts (e.g., conflicting data from different image data sets), setting thresholds for scoring routines, or the like. The linked components of the system can communicate with one another. This allows different systems disclosed herein to exchange information, transform data, and process data to dynamically update surgical plans, generative new surgical plans, or the like.

In some embodiments, the display 122 may include one or more selection features enabling a user (e.g., a surgeon) to select one of the displayed surgical plans to proceed with (e.g., as described with respect to the block 326 of the method 320 of FIG. 3B). For example, the display 122 includes a first selection feature 428 (e.g., a first selection button) that the user selects if they desire to proceed with the first surgical plan 400, and a second selection feature 528 (e.g., a second selection button) that the user selects if they desire to proceed with the second surgical plan 500. Spinal implants can be configured for use with either the first surgical procedure of the first surgical plan 400 or a second surgical procedure of the first surgical plan 500. The implants can be designed based at least in part on the corresponding virtual model by, for example, generating virtual implant models that fit the corresponding virtual model. In some embodiments, the anatomical elements of a virtual model can be selected for designing the implants. A negative space between the selected anatomical elements can be filled with a virtual three-dimensional implant using one or more negative space filling routines. The system disclosed herein can select the footprint or design constraints for the implant. In other embodiments, a user can input footprints, design constraints, and other information as a design parameter(s).

Although only two surgical plans are illustrated in FIG. 5, in some embodiments more than two surgical plans (e.g., three, four, five, etc.) are generated and transmitted for surgeon review. In such embodiments, the display 122 can display each surgical plan for side-by-side comparison. In some embodiments, the display 122 and the surgical plans are interactive, such that the user can also quickly and easily toggle between the different surgical plans. This is expected to be useful in embodiments in which each surgical plan does not fit on a single display screen. In some embodiments, the user can select various like features of the multiple surgical plans to compare side-by-side (e.g., the user can select to have a side-be-side view of virtual models of the predicted post-op patient anatomy associated with each of the surgical plans).

### C. Additional Select Methods and Systems for Modeling and Designing Patient-Specific Surgical Plans

FIG. 6 is a flow diagram illustrating a method 600 for generating a surgical plan, according to an embodiment of the present technology. In some embodiments, the method 600 is performed at blocks 302 and 304 of the method 300 described previously. The method 600 can include a data phase 610 and a modeling phase 620. The data phase 610 can include collecting data of a patient to be treated (e.g., pathology data), and comparing the patient data to reference data (e.g., prior patient data such as pathology, surgical, and/or outcome data). For example, a patient data set can be received (block 612). The patient data set can be compared to a plurality of reference patient data sets (block 614), e.g., in order to identify one or more similar patient data sets in the plurality of reference patient data sets. Each of the plurality of reference patient data sets can include data representing one or more of age, gender, BMI, lumbar lordosis, Cobb angle(s), pelvic incidence, disc height, segment flexibility, bone quality, rotational displacement, or treatment level of the spine.

A subset of the plurality of reference patient data sets can be selected (block 616), e.g., based on similarity to the patient data set and/or treatment outcomes of the corresponding reference patients. For example, a similarity score can be generated for each reference patient data set, based on the comparison of the patient data set and the reference patient data set. The similarity score can represent a statistical correlation between the patient data and the reference patient data set. One or more similar patient data sets can be identified based, at least partly, on the similarity score.

In some embodiments, each patient data set of the selected subset includes and/or is associated with data indicative of a favorable treatment outcome (e.g., a favorable treatment outcome based on a single target outcome, aggregate outcome score, outcome thresholding). The data can include, for example, data representing one or more of corrected anatomical metrics, presence of fusion, health related quality of life, activity level, or complications. In some embodiments, the data is or includes an outcome score, which can be calculated based on a single target outcome, an aggregate outcome, and/or an outcome threshold.

Optionally, the data analysis phase 610 can include identifying or determining, for at least one patient data set of the selected subset (e.g., for at least one similar patient data set), surgical procedure data and/or medical device design data associated with the favorable treatment outcome. The surgical procedure data can include data representing one or more of a surgical approach, a corrective maneuver, a bony resection, or implant placement. The at least one medical device design can include data representing one or more of physical properties, mechanical properties, or biological properties of a corresponding medical device. In some embodiments, the at least one patient-specific medical device design includes a design for an implant or an implant delivery instrument.

In the modeling phase 620, a surgical plan is generated (block 622). The generating step can include developing at least one predictive model based on the patient data set and/or selected subset of reference patient data sets (e.g., using statistics, machine learning, neural networks, AI, or the like). The predictive model can be configured to generate surgical plan. The surgical plan can be generally similar to any of the surgical plans described herein.

In some embodiments, the predictive model includes one or more trained machine learning models that generate, at least partly, the surgical plan. For example, the trained machine learning model(s) can determine a plurality of candidate surgical plans for treating the patient. Each surgical plan can be associated with a corresponding medical device design. In some embodiments, the surgical plans and/or medical device designs are determined based on surgical procedure data and/or medical device design data associated with favorable outcomes, as previously described with respect to the data analysis phase 610. For each surgical plan and/or corresponding medical device design, the trained machine learning model(s) can calculate a probability of achieving a target outcome (e.g., favorable or desired outcome) for the patient. The trained machine learning model(s) can then select one or more surgical plans and/or corresponding medical device designs based, at least partly, on the calculated probabilities.

The method 600 can be implemented and performed in various ways. In some embodiments, one or more operations of the method 600 (e.g., the data phase 610 and/or the modeling phase 620) can be implemented as computer-readable instructions stored in memory and executable by one or more processors of any of the computing devices and systems described herein (e.g., the system 100), or a component thereof (e.g., the client computing device 102 and/or the server 106). Alternatively, one or more operations of the method 600 can be performed by a healthcare provider (e.g., physician, surgeon), a robotic apparatus (e.g., a surgical robot), or a combination thereof.

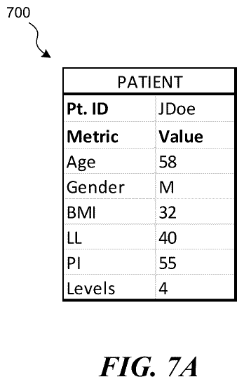

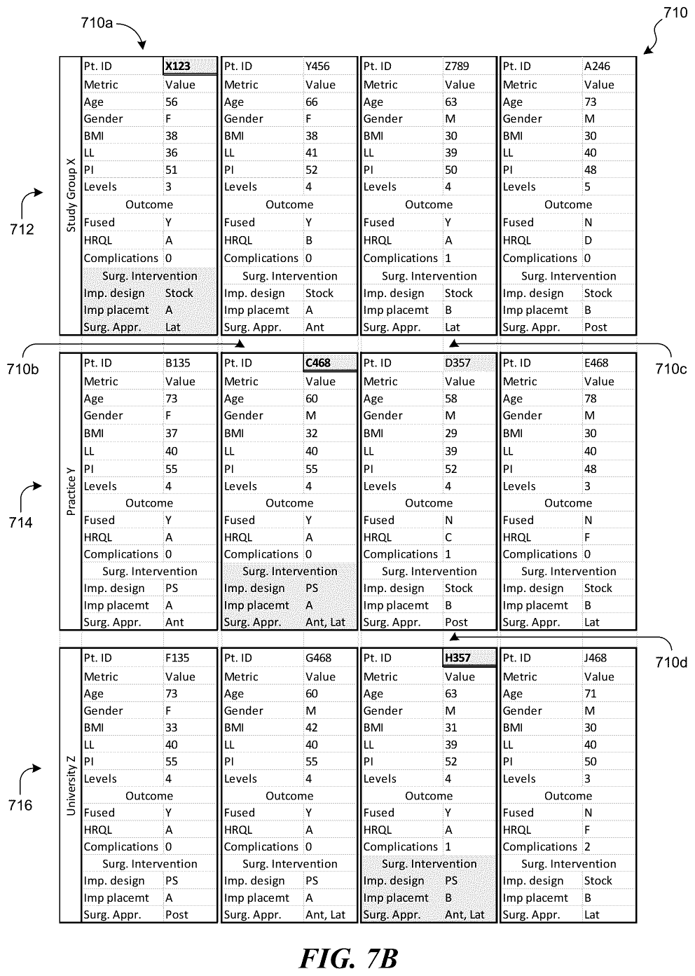

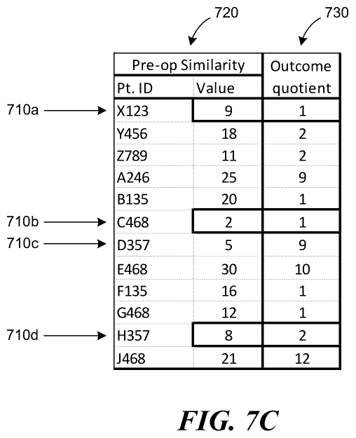

FIGS. 7A-7C illustrate exemplary data sets that may be used and/or generated in connection with the methods described herein (e.g., the data analysis phase 610 described with respect to FIG. 6), according to an embodiment of the present technology. FIG. 7A illustrates a patient data set 700 of a patient to be treated. The patient data set 700 can include a patient ID and a plurality of pre-operative patient metrics (e.g., age, gender, BMI, lumbar lordosis (LL), pelvic incidence (PI), and treatment levels of the spine (levels)). FIG. 7B illustrates a plurality of reference patient data sets 710. In the depicted embodiment, the reference patient data sets 710 include a first subset 712 from a study group (Study Group X), a second subset 717 from a practice database (Practice Y), and a third subset 716 from an academic group (University Z). In alternative embodiments, the reference patient data sets 710 can include data from other sources, as previously described herein. Each reference patient data set can include a patient ID, a plurality of pre-operative patient metrics (e.g., age, gender, BMI, lumbar lordosis (LL), pelvic incidence (PI), and treatment levels of the spine (levels)), treatment outcome data (Outcome) (e.g., presence of fusion (fused), HRQL, complications), and treatment procedure data (Surg. Intervention) (e.g., implant design, implant placement, surgical approach).

FIG. 7C illustrates comparison of the patient data set 700 to the reference patient data sets 710. As previously described, the patient data set 700 can be compared to the reference patient data sets 710 to identify one or more similar patient data sets from the reference patient data sets. In some embodiments, the patient metrics from the reference patient data sets 710 are converted to numeric values and compared the patient metrics from the patient data set 700 to calculate a similarity score 720 (“Pre-op Similarity”) for each reference patient data set. Reference patient data sets having a similarity score below a threshold value can be considered to be similar to the patient data set 700. For example, in the depicted embodiment, reference patient data set 710a has a similarity score of 9, reference patient data set 710b has a similarity score of 2, reference patient data set 710c has a similarity score of 5, and reference patient data set 710d has a similarity score of 8. Because each of these scores are below the threshold value of 10, reference patient data sets 710a-d are identified as being similar patient data sets.

The treatment outcome data of the similar patient data sets 710a-d can be analyzed to determine surgical plans and/or implant designs with the highest probabilities of success. For example, the treatment outcome data for each reference patient data set can be converted to a numerical outcome score 730 (“Outcome Quotient”) representing the likelihood of a favorable outcome. In the depicted embodiment, reference patient data set 710a has an outcome score of 1, reference patient data set 710b has an outcome score of 1, reference patient data set 710c has an outcome score of 9, and reference patient data set 710d has an outcome score of 2. In embodiments where a lower outcome score correlates to a higher likelihood of a favorable outcome, reference patient data sets 710a, 710b, and 710d can be selected. The treatment procedure data from the selected reference patient data sets 710a, 710b, and 710d can then be used to determine at least one surgical plan (e.g., implant placement, surgical approach) and/or implant design that is likely to produce a favorable outcome for the patient to be treated.

In some embodiments, a method for providing medical care to a patient is provided. The method can include comparing a patient data set to reference data. The patient data set and reference data can include any of the data types described herein. The method can include identifying and/or selecting relevant reference data (e.g., data relevant to treatment of the patient, such as data of similar patients and/or data of similar treatment procedures), using any of the techniques described herein. A surgical plan can be generated based on the selected data, using any of the techniques described herein. The surgical plan can include one or more treatment procedures (e.g., surgical procedures, instructions for procedures, models or other virtual representations of procedures), one or more medical devices (e.g., implanted devices, instruments for delivering devices, surgical kits), or a combination thereof.

In some embodiments, a system for generating a medical treatment plan is provided. The system can compare a patient data set to a plurality of reference patient data sets, using any of the techniques described herein. A subset of the plurality of reference patient data sets can be selected, e.g., based on similarity and/or treatment outcome, or any other technique as described herein. A surgical plan can be generated based at least in part on the selected subset, using any of the techniques described herein. The surgical plan can include one or more treatment procedures, one or more medical devices, or any of the other aspects of a treatment plan described herein, or combinations thereof.

In further embodiments, a system is configured to use historical patient data. The system can select historical patient data to develop or select a surgical plan, design medical devices, or the like. Historical data can be selected based on one or more similarities between the present patient and prior patients to develop a prescriptive treatment plan designed for desired outcomes. The prescriptive treatment plan can be tailored for the present patient to increase the likelihood of the desired outcome. In some embodiments, the system can analyze and/or select a subset of historical data to generate one or more surgical procedures, one or more medical devices, or a combination thereof. In some embodiments, the system can use subsets of data from one or more groups of prior patients, with favorable outcomes, to produce a reference historical data set used to, for example, design, develop or select the treatment plan, medical devices, or combinations thereof.

FIG. 8 is a flow diagram illustrating another method 800 for providing patient-specific medical care, according to another embodiment of the present technology. Similar to the method 600 described with reference to FIG. 6, the method 800 can be performed at blocks 302 and 304 of the method 300 described previously.

The method 800 can begin in block 802 by receiving a patient data set for a particular patient in need of medical treatment. In some embodiments, block 802 can be the same as or generally similar to the block 302 of the method 300 of FIG. 3A. For example, receiving a patient data set can include receiving patient image data, patient metrics, or any of the other patient data described herein.

Once the patient data set is received in block 802, the method 800 can continue in block 803 by creating a virtual model of the patient's native anatomical configuration (also referred to as “pre-operative anatomical configuration”). The virtual model can be based on the image data included in the patient data set received in block 802. For example, the same computing system that received the patient data set in block 802 can analyze the image data in the patient data set to generate a virtual model of the patient's native anatomical configuration. The virtual model can be a two- or three-dimensional visual representation of the patient's native anatomy. The virtual model can include one or more regions of interest, and may include some or all of the patient's anatomy within the regions of interest (e.g., any combination of tissue types including, but not limited to, bony structures, cartilage, soft tissue, vascular tissue, nervous tissue, etc.). As a non-limiting example, the virtual model can include a visual representation of the patient's spinal cord region, including some or all of the sacrum, lumbar region, thoracic region, and/or cervical region. In some embodiments, the virtual model includes soft tissue, cartilage, and other non-bony structures. In other embodiments, the virtual model only includes the patient's bony structures. In some embodiments, the method 800 can optionally omit creating a virtual model of the patient's native anatomy in block 803, and proceed directly from block 802 to block 804.

In some embodiments, the computing system that generated the virtual model in block 802 can also determine (e.g., automatically determine or measure) one or more disease metrics of the patient based on the virtual model. For example, the computing system may analyze the virtual model to determine the patient's pre-operative lumbar lordosis, Cobb angles, coronal parameters (e.g., coronal balance, global coronal balance, coronal pelvic tilt, etc.), sagittal parameters (e.g., pelvic incidence, sacral slope, thoracic kyphosis, etc.) and/or pelvic parameters. The disease metrics can include micro-measurements (e.g., metrics associated with specific or individual segments of the patient's spine) and/or macro-measurements (e.g., metrics associated with multiple segments of the patient's spine).

The method 800 can continue in block 804 by creating a virtual model of a corrected anatomical configuration (which can also be referred to herein as the “planned configuration,” “optimized geometry,” “post-operative anatomical configuration,” or “target outcome”) for the patient. For example, the computing system can, using the analysis procedures described previously, determine a “corrected” or “optimized” anatomical configuration for the particular patient that represents an ideal surgical outcome for the particular patient. This can be done, for example, by analyzing a plurality of reference patient data sets to identify post-operative anatomical configurations for similar patients who had a favorable post-operative outcome, as previously described in detail with respect to FIGS. 6-7C (e.g., based on similarity of the reference patient data set to the patient data set and/or whether the reference patient had a favorable treatment outcome). This may also include applying one or more mathematical rules defining optimal anatomical outcomes (e.g., positional relationships between anatomic elements) and/or target (e.g., acceptable) post-operative metrics/design criteria (e.g., adjust anatomy so that the post-operative sagittal vertical axis is less than 7 mm, the post-operative Cobb angle less than 10 degrees, etc.). Target post-operative metrics can include, but are not limited to, target coronal parameters, target sagittal parameters, target pelvic incidence angle, target Cobb angle, target shoulder tilt, target iliolumbar angle, target coronal balance, target Cobb angle, target lordosis angle, and/or a target intervertebral space height. The difference between the native anatomical configuration and the corrected anatomical configuration may be referred to as a “patient-specific correction” or “target correction.”

Once the corrected anatomical configuration is determined, the computing system can generate a two- or three-dimensional visual representation of the patient's anatomy with the corrected anatomical configuration. As with the virtual model created in block 803, the virtual model of the patient's corrected anatomical configuration can include one or more regions of interest, and may include some or all of the patient's anatomy within the regions of interest (e.g., any combination of tissue types including, but not limited to, bony structures, cartilage, soft tissue, vascular tissue, nervous tissue, etc.). As a non-limiting example, the virtual model can include a visual representation of the patient's spinal cord region in a corrected anatomical configuration, including some or all of the sacrum, lumbar region, thoracic region, and/or cervical region. In some embodiments, the virtual model includes soft tissue, cartilage, and other non-bony structures. In other embodiments, the virtual model only includes the patient's bony structures.

The method 800 can continue in block 806 by generating (e.g., automatically generating) a surgical plan for achieving the corrected anatomical configuration shown by the virtual model. The surgical plan can be generally similar to the surgical plans described herein, and can include, for example, pre-operative plans, operative plans, post-operative plans, and/or specific spine metrics associated with the optimal surgical outcome. For example, the surgical plans can include a specific surgical procedure for achieving the corrected anatomical configuration. In the context of spinal surgery, the surgical plan may include a specific fusion surgery (e.g., PLIF, ALIF, TLIF, LLIF, DLIF, XLIF, etc.) across a specific range of vertebral levels (e.g., L1-L4, L1-5, L3-T12, etc.). Of course, other surgical procedures may be identified for achieving the corrected anatomical configuration, such as non-fusion surgical approaches and orthopedic procedures for other areas of the patient. The surgical plan may also include one or more expected spine metrics (e.g., lumbar lordosis, Cobb angles, coronal parameters, sagittal parameters, and/or pelvic parameters) corresponding to the expected post-operative patient anatomy, as previously described. The surgical plan can be generated by the same or different computing system that created the virtual model of the corrected anatomical configuration. In some embodiments, the surgical plan can also be based on one or more reference patient data sets as previously described with respect to FIGS. 6-7C. In some embodiments, the surgical plan can also be based at least in part on surgeon-specific preferences and/or outcomes associated with a specific surgeon performing the surgery. As described above with respect to FIG. 3A, in some embodiments, more than one surgical plan is generated in block 806 to provide a surgeon with multiple options.

FIG. 9 is a schematic illustration of an operative setup including select systems and devices that can be used to provide patient-specific medical care, such as for performing certain operations described above with respect to the methods 300, 320, 600, and 800, described with respect to FIGS. 3A, 3B, 6, and 8, respectively. As shown, the operative setup includes a computing device 902, a computing system 906, a cloud 908, a manufacturing system 930, and a robotic surgical platform 950. The computing device 902 can be a user device, such as a smart phone, mobile device, laptop, desktop, personal computer, tablet, phablet, or other such devices known in the art. In operation, a user (e.g., a surgeon) can collect, retrieve, review, modify, or otherwise interact with a patient data set using the computing device 902. The computing system 906 can include any suitable computing system configured to store one or more software modules for identifying reference patient data sets, determining patient-specific surgical plans, generating virtual models of patient anatomy, designing patient-specific implants, or the like. The one or more software modules can include algorithms, machine-learning models, artificial intelligence architectures, or the like for performing select operations. The cloud 908 can be any suitable network and/or storage system, and may include any combination of hardware and/or virtual computing resources. The manufacturing system 930 can be any suitable manufacturing system for producing patient-specific implants, including any of those previously described herein. The robotic surgical platform 950 (referred to herein as “the platform 950”) can be configured to perform or otherwise assist with one or more aspects of a surgical procedure.

In a representative operation, the computing device 902, the computing system 906, the cloud 908, the manufacturing system 930, and the platform 950 can be used to provide patient-specific medical care, such as to perform the methods described herein. For example, the computing system 906 can receive a patient data set from the computing device 902 (e.g., block 302 of the method 300, block 802 of the method 800, etc.). In some embodiments, the computing device 902 can directly transmit the patient data set to the computing system 906. In other embodiments, the computing device 902 can upload the patient data set into the cloud 908, and the computing system 906 can download or otherwise access the patient data set from the cloud. Once the computing system 906 receives the patient data set, the computing system 906 can create a virtual model of the patient's native anatomical configuration (e.g., block 803 of the method 800), create a virtual model of the corrected anatomical configuration (e.g., block 804 of the method 800), and/or generate a surgical plan for achieving the corrected anatomical configuration (e.g., block 304 of the method 300, block 806 of the method 800, etc.). The computing system can perform the foregoing operations via the one or more software modules, which in some embodiments include machine learning models or other artificial intelligence architectures. Once the surgical plans, including any associated virtual models, are created, the computing system 906 can transmit the surgical plans to the surgeon for review (e.g., block 306 of the method 300). This can include, for example, directly transmitting the surgical plans to the computing device 902 for surgeon review. In other embodiments, this can include uploading the virtual models and the surgical plan to the cloud 908. A surgeon can then download or otherwise access the virtual models and the surgical plan from the cloud 908 using the computing device 902.

The surgeon can use the computing device 902 to review the surgical plans, as described previously. The surgeon can also approve or reject the surgical plans and provide any feedback regarding the surgical plans using the computing device 902. The surgeon's approval, rejection, and/or feedback regarding the surgical plan can be transmitted to, and received by, the computing system 906 (e.g., blocks 308 and 310 of the method 300). The computing system 906 can than revise the virtual surgical plans and associated virtual models (e.g., block 312 of the method 300). The computing system 906 can transmit the revised virtual model and surgical plan to the surgeon for review (e.g., by uploading it to the cloud 908 or directly transmitting it to the computing device 902).

The computing system 906 can also design the patient-specific implant based on the selected surgical plan (e.g., block 314 of the method 300) using, the one or more software modules. In some embodiments, software modules rely on one or more algorithms, machine learning models, or other artificial intelligence architectures to design the implant. Once the computing system 906 designs the patient-specific implant, the computing system 906 can upload the design and/or manufacturing instructions to the cloud 908. The computing system 906 may also create fabrication instructions (e.g., computer-readable fabrication instructions) for manufacturing the patient-specific implant. In such embodiments, the computing system 906 can upload the fabrication instructions to the cloud 908.

The manufacturing system 930 can download or otherwise access the design and/or fabrication instructions for the patient-specific implant from the cloud 908. The manufacturing system can then manufacture the patient-specific implant (e.g., block 316 in the method 300) using additive manufacturing techniques, subtractive manufacturing techniques, or other suitable manufacturing techniques.

The robotic surgical platform 950 can perform or otherwise assist with one or more aspects of the surgical procedure (e.g., block 318 of the method 300). For example, the platform 950 can prepare tissue for an incision, make an incision, make a resection, remove tissue, manipulate tissue, perform a corrective maneuver, deliver the implant to a target site, deploy the implant at the target site, adjust the implant at the target site, manipulate the implant once it is implanted, secure the implant at the target site, explant the implant, suture tissue, etc. The platform 950 can therefore include one or more arms 955 and end effectors for holding various surgical tools (e.g., graspers, clips, needles, needle drivers, irrigation tools, suction tools, staplers, screw driver assemblies, etc.), imaging instruments (e.g., cameras, sensors, etc.), and/or medical devices (e.g., the implant 900) and that enable the platform 950 to perform the one or more aspects of the surgical plan. Although shown as having one arm 955, one skilled in the art will appreciate that the platform 950 can have a plurality of arms (e.g., two, three, four, or more) and any number of joints, linkages, motors, and degrees of freedom. In some embodiments, the platform 950 may have a first arm dedicated to holding one or more imaging instruments, while the remainder of the arms hold various surgical tools. In some embodiments, the tools can be releasably secured to the arms such that they can be selectively interchanged before, during, or after an operative procedure. The arms can be moveable through a variety of ranges of motion (e.g., degrees of freedom) to provide adequate dexterity for performing various aspects of the operative procedure.

The platform 950 can include a control module 960 for controlling operation of the arm(s) 955. In some embodiments, the control module 960 includes a user input device (not shown) for controlling operation of the arm(s) 955. The user input device can be a joystick, a mouse, a keyboard, a touchscreen, an infrared sensor, a touchpad, a wearable input device, a camera- or image-based input device, a microphone, or other user input devices. A user (e.g., a surgeon) can interact with the user input device to control movement of the arm(s) 955. A patient account can be linked to the surgical procedure being performed on the patient P to enable real-time intra-operative viewing of simulated post-operative metrics for the surgical procedure. This allows the surgical team to compare real-time imaging (e.g., X-rays, fluoroscopy imaging, etc.) to the simulated post-operative anatomy. Implants can be repositioned any number of times to achieve the target anatomical configuration corresponding to the target post-operative metrics. Additional intra-operative simulations can be generated based on intra-operative data (e.g., intra-operative images) to generate modified or new surgical plans or steps. The computing system 906 can update the virtual anatomical model of the patient with the real-time intra-operative data to generate updated post-operative spinal metrics of the at least one virtual anatomical model in the post-operative configuration for the surgical procedure. The intra-operative data can trigger changes to the surgical procedure based on the updated post-operative spinal metrics.

In some embodiments, the control module 960 includes one or more processors for executing machine-readable operative instructions that, when executed, automatically control operation of the arm 955 to perform one or more aspects of the surgical procedure. In some embodiments, the control module 960 may receive the machine-readable operative instructions (e.g., from the cloud 908) specifying one or more steps of the surgical procedure that, when executed by the control module 960, cause the platform 950 to perform the one or more steps of the surgical procedure. For example, the machine-readable operative instructions may direct the platform 950 to prepare tissue for an incision, make an incision, make a resection, remove tissue, manipulate tissue, perform a corrective maneuver, deliver the implant 900 to a target site, deploy the implant 900 at the target site, adjust a configuration of the implant 900 at the target site, manipulate the implant 900 once it is implanted, secure the implant 900 at the target site, explant the implant 900, suture tissue, and the like. The operative instructions may therefor include particular instructions for articulating the arm 955 to perform or otherwise aid in the delivery of the patient-specific implant.

In some embodiments, the platform 950 can generate (e.g., as opposed to simply receiving) the machine-readable operative instructions based on the surgical plan. For example, the surgical plan can include information about the delivery path, tools, and implantation site. The platform 950 can analyze the surgical plan and develop executable operative instructions for performing the patient-specific procedure based on the capabilities (e.g., configuration and number of robotic arms, functionality of and effectors, guidance systems, visualization systems, etc.) of the robotic system. This enables the operative setup shown in FIG. 9 to be compatible with a wide range of different types of robotic surgery systems.

The platform 950 can include one or more communication devices (e.g., components having VLC, WiMAX, LTE, WLAN, IR communication, PSTN, Radio waves, Bluetooth, and/or Wi-Fi operability) for establishing a connection with the cloud 908 and/or the computing device 902 for accessing and/or downloading the surgical plan and/or the machine-readable operative instructions. For example, the cloud 908 can receive a request for a particular surgical plan from the platform 950 and send the plan to the platform 950. Once identified, the cloud 908 can transmit the surgical plan directly to the platform 950 for execution. In some embodiments, the cloud 908 can transmit the surgical plan to one or more intermediate networked devices (e.g., the computing device 902) rather than transmitting the surgical plan directly to the platform 950. A user can review the surgical plan using the computing device 902 before transmitting the surgical plan to the platform 950 for execution. Additional details for identifying, storing, downloading, and accessing patient-specific surgical plans are described in U.S. application Ser. No. 19/960,810, filed Aug. 11, 2020, the disclosure of which is incorporated by reference herein in its entirety.

The platform 950 can include additional components not expressly shown in FIG. 9. For example, in various embodiments the platform 950 may include one or more displays (e.g., LCD display screen, an LED display screen, a projected, holographic, or augmented reality display (e.g., a heads-up display device or a head-mounted device), one or more I/O devices (e.g., a network card, video card, audio card, USB, firewire or other external device, camera, printer, speakers, CD-ROM drive, DVD drive, disk drive, or Blu-Ray device), and/or a memory (e.g., random access memory (RAM), various caches, CPU registers, read-only memory (ROM), and writable non-volatile memory, such as flash memory, hard drives, floppy disks, CDs, DVDs, magnetic storage devices, tape drives, device buffers, and so forth). In some embodiments, the foregoing components can be generally similar to the like components described in detail with respect to computing device 200 in FIG. 2.

Without being bound by theory, using a robotic surgical platform to perform various aspects of the surgical plans described herein is expected to provide several advantages over conventional operative techniques. For example, use of robotic surgical platforms may improve surgical outcomes and/or shorten recovery times by, for example, decreasing incision size, decreasing blood loss, decreasing a length of time of the operative procedure, increasing the accuracy and precision of the surgery (e.g., the placement of the implant at the target location), and the like. The platform 950 can also avoid or reduce user input errors, e.g., by including one or more scanners for obtaining information from instruments (e.g., instruments with retrieval features), tools, the patient specific implant 900 (e.g., after the implant 900 has been gripped by the arm 955), etc. The platform 950 can confirm use of proper instruments prior and during the surgical procedure. If the platform 950 identifies an incorrect instrument or tool, an alert can be sent to a user that another instrument or tool should be installed. The user can scan the new instrument to confirm that the instrument is appropriate for the surgical plan. In some embodiments, the surgical plan includes instructions for use, a list of instruments, instrument specifications, replacement instruments, and the like. The platform 950 can perform pre- and post-surgical checking routines based on information from the scanners.

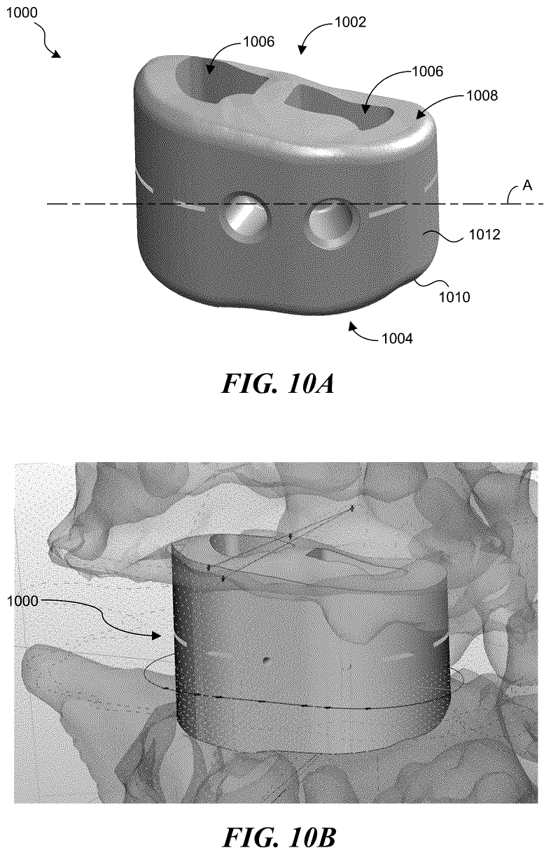

FIG. 10A illustrates an example of a patient-specific implant 1000 (e.g., as designed in block 314 and manufactured in block 316 of the method 300), and FIG. 10B illustrates the implant 1000 implanted in the patient. The implant 1000 can be any orthopedic or other implant specifically designed to induce the patient's body to conform to the previously identified corrected anatomical configuration. In the illustrated embodiment, the implant 1000 is a vertebral interbody device having a first (e.g., upper) surface 1002 configured to engage an inferior endplate surface of a superior vertebral body, a second (e.g., lower) surface 1004 configured to engage a superior endplate surface of an inferior vertebral body, and a main body 1012.

The implant 1000 can include one or more patient-specific or custom surfaces. For example, the first (superior) surface 1002 can have a first patient-specific topography or shape 1008, the second (inferior) surface 1004 can have a second patient-specific topography or shape 1010, and the main body 1012 can be configured to received material (e.g., bone material, autographs, allografts, bone matrices, etc.) configured to promote spinal fusion. In some surgical procedures, the main body 1012 can be filled with material immediately prior to insertion into the patient. After implantation, the material can interact with the surrounding anatomical features (e.g., vertebral bodies, endplates, etc.) to promote fusion between the implant and the adjacent bony tissue. If a virtual anatomical model is used to design the implant 100, the superior patient-specific topography or shape 1008 of the superior patient-specific surface 1002 can generally match a first vertebral surface of the at least one virtual anatomical model, and the inferior superior patient-specific topography or shape 1010 of the inferior patient-specific surface 1004 can generally match a second vertebral surface of the at least one virtual anatomical model. That is, the first surface 1002 can have a patient-specific topography designed to match (e.g., mate with) the topography of the inferior endplate surface of the superior vertebral body to form a generally gapless interface therebetween. Likewise, the second surface 1004 can have a patient-specific topography designed to match or mate with the topography of the superior endplate surface of the inferior vertebral body to form a generally gapless interface therebetween. This allows for custom fitting with the patient's anatomy, as shown in FIG. 10B.

The implant 1000 may also include one or more receiving-features 1006 (e.g., holes, cavities, aa recesses 1006 or other features in the main body 1012) configured to receive and hold material (that promotes bony ingrowth). The number, size, and positions of the receiving-features 1006 can be selected based on the configuration of the implant 1000 and spinal procedure. The systems disclosed herein can analyze the desired characteristics of the fusion procedure to design the main body 1012 of the implant to hold material. After implantation, the material can promote fusion with the bony tissue contacting and overlying the surfaces 1002, 1004.

Because the implant 1000 is patient-specific and designed to induce a geometric change in the patient, the implant 1000 is not necessarily symmetric, and is often asymmetric. For example, in the illustrated embodiment, the implant 1000 has a non-uniform thickness such that a plane defined by the first surface 1002 is not parallel to a central longitudinal axis A of the implant 1000. Of course, because the implants described herein, including the implant 1000, are patient-specific, the present technology is not limited to any particular implant design or characteristic. Additional features of patient-specific implants that can be designed and manufactured in accordance with the present technology are described in U.S. patent application Ser. Nos. 16/987,111 and 17/100,396, the disclosures of which are incorporated by reference herein in their entireties.

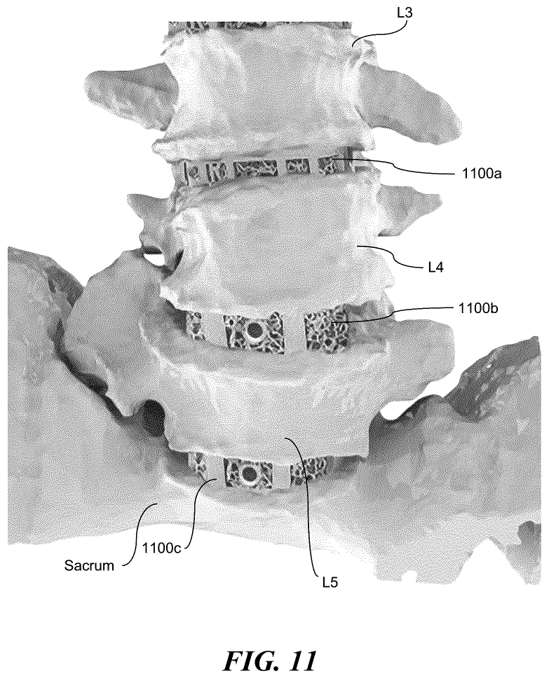

The patient-specific medical procedures described herein can involve implanting more than one patient-specific implant into the patient to achieve the corrected anatomical configuration (e.g., a multi-site procedure). FIG. 11, for example, illustrates a lower spinal cord region having three patient specific implants 1100a-1100c implanted at different vertebral levels. More specifically, a first implant 1100a is implanted between the L3 and L4 vertebral bodies, a second implant 1100b is implanted between the L4 and L5 vertebral bodies, and a third implant 1100c is implanted between the L5 vertebral body and the sacrum. Together, the implants 1100a-c can cause the patient's spinal cord region to assume the previously identified corrected anatomical configuration (e.g., transforming the patient's anatomy from its pre-operative diseased configuration to the post-operative optimized configuration). In some embodiments, more or fewer implants are used to achieve the corrected anatomical configuration. For example, in some embodiments one, two, four, five, six, seven, eight, or more implants are used to achieve the corrected anatomical configuration. In embodiments involving more than one implant, the implants do not necessarily have the same shape, size, or function. In fact, the multiple implants will often have different geometries and topographies to correspond to the target vertebral level at which they will be implanted. As also shown in FIG. 11, the patient-specific medical procedures described herein can involve treating the patient at multiple target regions (e.g., multiple vertebral levels).

As one skilled in the art will appreciate, any of the software modules described previously may be combined into a single software module for performing the operations described herein. Likewise, the software modules can be distributed across any combination of the computing systems and devices described herein, and are not limited to the express arrangements described herein. Accordingly, any of the operations described herein can be performed by any of the computing devices or systems described herein, unless expressly noted otherwise.

### Examples

The present technology is illustrated, for example, according to various aspects described below. Various examples of aspects of the present technology are described as numbered examples (1, 2, 3, etc.) for convenience. These are provided as examples and do not limit the present technology. It is noted that any of the dependent examples can be combined in any suitable manner, and placed into a respective independent example. The other examples can be presented in a similar manner.

1. An implant system comprising:
an intervertebral implant configured for implantation in a patient, the intervertebral implant including a superior patient-specific surface, an inferior patient-specific surface, and a main body configured to receive material configured to promote spinal fusion;
a first predicted post-operative three-dimensional representation and/or virtual model of patient anatomy associated with a first surgical procedure and including a first set of predicted post-operative spinal metrics; and
a second predicted post-operative three-dimensional representation and/or virtual model of patient anatomy associated with a second surgical procedure and including a second set of predicted post-operative spinal metrics,
wherein the intervertebral implant is configured to be implanted in conjunction with either the first surgical procedure or the second surgical procedure, and wherein the intervertebral implant is designed based at least in part on the corresponding predicted post-operative three-dimensional representation and/or virtual model.

2. The implant system of example 1 wherein the superior patient-specific surface of the intervertebral implant matches a first vertebral surface of the corresponding post-operative three-dimensional representation and/or virtual model, and the inferior patient-specific surface of the intervertebral implant matches a second vertebral surface of the corresponding post-operative three-dimensional representation and/or virtual model.

3. The implant system of example 1, further comprising one or more implants configured to be implanted in conjunction with the intervertebral implant, wherein the one or more implants are designed based at least in part on the corresponding post-operative three-dimensional representation and/or virtual model.

4. The implant system of example 1 wherein the first predicted post-operative three-dimensional representation or virtual model is linked to an interactive surgical planner configured to receive at least one physician post-operative metric, wherein the first predicted post-operative three-dimensional representation or virtual model is configured to be modified to meet the at least one physician post-operative metric and to be measured for obtaining a modified first set of predicted post-operative spinal metrics based on the modified post-operative three-dimensional representation or virtual model.

5. The implant system of example 1 wherein the first predicted post-operative three-dimensional representation or virtual model is linked to an account for the patient and configured for post-operative metric measuring, wherein the account includes simulated post-operative metrics for at least the first surgical procedure and the second surgical procedure.

6. The implant system of example 1 wherein an account for the patient is linked to a surgical procedure being performed on the patient to enable real-time intra-operative viewing of simulated post-operative metrics for the surgical procedure.

7. The implant system of example 1 wherein the first predicted post-operative three-dimensional representation and/or virtual model and the second predicted post-operative three-dimensional representation and/or virtual model include repositionable spinal anatomical elements.

8. The implant system of example 7 wherein:
the first predicted post-operative three-dimensional representation and/or virtual model includes first model data for measuring the first set of predicted post-operative spinal metrics in a first predicted post-operative configuration associated with performance of the first surgical procedure, and
the second predicted post-operative three-dimensional representation and/or virtual model includes second model data for measuring the second set of predicted post-operative spinal metrics in a second predicted post-operative configuration associated with performance of the second surgical procedure.

9. The implant system of example 8 wherein the first model data and/or the second model data include surface rendering data, measured patient metrics, and/or image data from images of the patient.

10. The implant system of example 1, further comprising an interactive surgical plan configured to concurrently display:
the first predicted post-operative three-dimensional representation and/or virtual model of patient anatomy and the first set of predicted post-operative spinal metrics; and
the second predicted post-operative three-dimensional representation and/or virtual model of patient anatomy and the second set of predicted post-operative spinal metrics.

11. The implant system of example 1, further comprising an interactive surgical plan configured to receive user input and to display dynamic comparisons of the first surgical procedure and the second surgical procedure based on user input.

12. The implant system of example 1, further comprising an interactive surgical planner visually comparing multiple surgical plans with predicted outcome data.

13. A computer-implemented method for providing patient-specific medical care to a patient, the method comprising:
generating a first surgical plan for the patient, the first surgical plan including:
   a first target location for surgical intervention,
   a first surgical procedure to be performed at the first target location, and
   a first virtual model of a first predicted post-operative anatomical configuration of the patient based on the first target location and the first surgical procedure, and/or first post-operative patient metrics associated with the first predicted post-operative anatomical configuration;
generating a second surgical plan for the patient that is different than the first surgical plan, the second surgical plan including:
   a second target location for surgical intervention,
   a second surgical procedure to be performed at the second target location, and
   a second virtual model of a second predicted post-operative anatomical configuration of the patient based on the second target location and the second surgical procedure, and/or second post-operative patient metrics associated with the second predicted post-operative anatomical configuration;
transmitting the first surgical plan and the second surgical plan for surgeon review; and
receiving a selection of either the first surgical plan or the second surgical plan, the selection providing an indication that the selected surgical plan is to be performed on the patient.

14. The computer-implemented method of example 13 wherein the first surgical procedure is different than the second surgical procedure.

15. The computer-implemented method of example 14 wherein the first surgical procedure includes spinal fusion, artificial disc replacement, vertebroplasty, kyphoplasty, spinal laminectomy, discectomy, facetectomy, or foraminotomy.

16. The computer-implemented method of example 15 wherein the first surgical procedure includes spinal fusion.

17. The computer-implemented method of example 14 wherein the first surgical procedure includes spinal fusion surgery, and wherein the second surgical procedure includes spinal non-fusion surgery.

18. The computer-implemented method of example 17 wherein:
the spinal fusion surgery includes one or more of posterior lumbar interbody fusion (PLIF), anterior lumbar interbody fusion (ALIF), transverse or transforaminal lumbar interbody fusion (TLIF), lateral lumbar interbody fusion (LLIF), direct lateral lumbar interbody fusion (DLIF), or extreme lateral lumbar interbody fusion (XLIF), and
the spinal non-fusion surgery includes artificial disc replacement.

19. The computer-implemented method of example 13 wherein the first surgical plan includes the first virtual model, and wherein the second surgical plan includes the second virtual model.

20. The computer-implemented method of example 19 wherein the first virtual model is a three-dimensional model of the first predicted post-operative anatomical configuration, and wherein the second virtual model is a three-dimensional model of the second predicted post-operative anatomical configuration, the first post-operative anatomical configuration being different than the second post-operative anatomical configuration.

21. The computer-implemented method of example 13 wherein the first surgical plan includes the first post-operative patient metrics, and wherein the second surgical plan includes the second post-operative patient metrics.

22. The computer-implemented method of example 21 wherein:
the first post-operative patient metrics include predicted values for one or more coronal parameters, sagittal parameters, pelvic parameters, Cobb angles, shoulder tilt, iliolumbar angles, coronal balance, lordosis angles, intervertebral space height, or other similar spinal or spinopelvic parameters associated with the first surgical plan, and
the second post-operative patient metrics include predicted values for one or more coronal parameters, sagittal parameters, pelvic parameters, Cobb angles, shoulder tilt, iliolumbar angles, coronal balance, lordosis angles, intervertebral space height, or other similar spinal or spinopelvic parameters associated with the second surgical plan.

23. The computer-implemented method of example 13 wherein the first surgical plan further includes a first predicted disease progression, and wherein the second surgical plan further includes a second predicted disease progression.

24. The computer-implemented method of example 13 wherein the first target location partially overlaps with, but is not the same as, the second target location.

25. The computer-implemented method of example 13 wherein the first target location is the same as the second target location.

26. The computer-implemented of example 13, further comprising designing a patient-specific implant based on the selected surgical plan.

27. The computer-implemented method of example 13 wherein generating the first surgical plan includes using a machine learning model to generate the first surgical plan, and wherein generating the second surgical plan includes using the machine learning model to generate the second surgical plan.

28. A computer-implemented method for providing patient-specific medical care to a patient, the method comprising:
displaying a first surgical plan for the patient, the first surgical plan including:
   a first target location for surgical intervention,
   a first surgical procedure to be performed at the first target location, and
   a first virtual model of a first predicted post-operative anatomical configuration of the patient based on the first target location and the first surgical procedure, and/or first post-operative patient metrics associated with the first predicted post-operative anatomical configuration;
displaying a second surgical plan for the patient that is different than the first surgical plan, the second surgical plan including:
   a second target location for surgical intervention,
   a second surgical procedure to be performed at the second target location, and
   a second virtual model of a second predicted post-operative anatomical configuration of the patient based on the second target location and the second surgical procedure, and/or second post-operative patient metrics associated with the second predicted post-operative anatomical configuration;
receiving a selection of the first surgical plan or the second surgical plan from a user, the selection providing an indication that the selected surgical plan is to be performed on the patient; and
transmitting the selected surgical plan.

29. The computer-implemented method of example 28 wherein the displaying the first surgical plan and displaying the second surgical plan includes simultaneously displaying at least part of the first surgical plan and at least part of the second surgical plan via a common display.

30. The computer-implemented method of example 29 wherein the first surgical plan and the second surgical plan are displayed side-by-side on the common display.

31. The computer-implemented method of example 28 wherein the first surgical procedure is different than the second surgical procedure.

32. The computer-implemented method of example 31 wherein the first surgical procedure includes spinal fusion, artificial disc replacement, vertebroplasty, kyphoplasty, spinal laminectomy, discectomy, facetectomy, or foraminotomy.

33. The computer-implemented method of example 32 wherein the first surgical procedure includes spinal fusion.

34. The computer-implemented method of example 31 wherein the first surgical procedure includes spinal fusion surgery, and wherein the second surgical procedure includes spinal non-fusion surgery.

35. The computer-implemented method of example 34 wherein:
the spinal fusion surgery includes one or more of posterior lumbar interbody fusion (PLIF), anterior lumbar interbody fusion (ALIF), transverse or transforaminal lumbar interbody fusion (TLIF), lateral lumbar interbody fusion (LLIF), direct lateral lumbar interbody fusion (DLIF), or extreme lateral lumbar interbody fusion (XLIF), and
the spinal non-fusion surgery includes artificial disc replacement.

36. The computer-implemented method of example 28 wherein the first surgical plan includes the first virtual model, and wherein the second surgical plan includes the second virtual model.

37. The computer-implemented method of example 36 wherein the first virtual model is a three-dimensional model of the first predicted post-operative anatomical configuration, and wherein the second virtual model is a three-dimensional model of the second predicted post-operative anatomical configuration, the first post-operative anatomical configuration being different than the second post-operative anatomical configuration.

38. The computer-implemented method of example 28 wherein the first surgical plan includes the first post-operative patient metrics, and wherein the second surgical plan includes the second post-operative patient metrics.

39. The computer-implemented method of example 38 wherein:
the first post-operative patient metrics include predicted values for one or more coronal parameters, sagittal parameters, pelvic parameters, Cobb angles, shoulder tilt, iliolumbar angles, coronal balance, lordosis angles, intervertebral space height, or other similar spinal parameters associated with the first surgical plan, and
the second post-operative patient metrics include predicted values for one or more coronal parameters, sagittal parameters, pelvic parameters, Cobb angles, shoulder tilt, iliolumbar angles, coronal balance, lordosis angles, intervertebral space height, or other similar spinal parameters associated with the second surgical plan.

40. The computer-implemented method of example 28 wherein the first surgical plan further includes a first predicted disease progression, and wherein the second surgical plan further includes a second predicted disease progression.

41. The computer-implemented method of example 28 wherein the first target location partially overlaps with, but is not the same as, the second target location.

42. The computer-implemented method of example 28 wherein the first target location is the same as the second target location.

43. A computer-implemented method for providing patient-specific medical care to a patient, the method comprising:
receiving user input for candidate procedures;
analyzing patient data of a patient to identify a plurality of candidate procedures based on the user input for candidate procedures;
for each of the identified candidate procedures, generating a respective surgical plan with a virtual post-operative anatomical model for the patient; and
generating a comparison of two or more of the surgical plans for viewing by a user using an electronic user device.

44. The computer-implemented method of example 43 wherein the user input for candidate procedures includes at least one of:
type of procedure,
one or more levels of the patient's spine,
payment information for the patient, or
one or more candidate procedure elimination criteria.

45. The computer-implemented method of example 43 wherein analyzing the patient data further comprises:
selecting one or more candidate procedure criteria based on the user input; and
using a machine learning model trained using prior patient data sets that meet the one or more candidate procedure criteria to identify the plurality of candidate procedures.

46. The computer-implemented method of example 43, further comprising transmitting the comparison for viewing, via the electronic user device, by the user, wherein the comparison includes one or more predicted post-operative metrics for each of the two or more of the surgical plans.

47. The computer-implemented method of example 43, further comprising:
determining a score for each of the surgical plans;
ranking the surgical plans based on the determined scores; and selecting a set of the surgical plans for the comparison based on the ranking.

48. The computer-implemented method of example 43, further comprising:
determining a score for each of the surgical plans;
selecting a set of the surgical plans each with scores above a threshold score; and comparing the surgical plans in the set for user review.

49. The computer-implemented method of example 48, wherein the threshold score is inputted by the user or determined based on the patient data.

### CONCLUSION

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In some embodiments, several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a floppy disk, a hard disk drive, a CD, a DVD, a digital tape, a computer memory, etc.; and a transmission type medium such as a digital and/or an analog communication medium (e.g., a fiber optic cable, a waveguide, a wired communications link, a wireless communication link, etc.).

Those skilled in the art will recognize that it is common within the art to describe devices and/or processes in the fashion set forth herein, and thereafter use engineering practices to integrate such described devices and/or processes into data processing systems. That is, at least a portion of the devices and/or processes described herein can be integrated into a data processing system via a reasonable amount of experimentation. Those having skill in the art will recognize that a typical data processing system generally includes one or more of a system unit housing, a video display device, a memory such as volatile and non-volatile memory, processors such as microprocessors and digital signal processors, computational entities such as operating systems, drivers, graphical user interfaces, and applications programs, one or more interaction devices, such as a touch pad or screen, and/or control systems including feedback loops and control motors (e.g., feedback for sensing position and/or velocity; control motors for moving and/or adjusting components and/or quantities). A typical data processing system may be implemented utilizing any suitable commercially available components, such as those typically found in data computing/communication and/or network computing/communication systems.

The herein described subject matter sometimes illustrates different components contained within, or connected with, different other components. It is to be understood that such depicted architectures are merely examples, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermediate components. Likewise, any two components so associated can also be viewed as being “operably connected,” or “operably coupled,” to each other to achieve the desired functionality, and any two components capable of being so associated can also be viewed as being “operably couplable” to each other to achieve the desired functionality. Specific examples of operably couplable include but are not limited to physically mateable and/or physically interacting components and/or wirelessly interactable and/or wirelessly interacting components and/or logically interacting and/or logically interactable components.

The embodiments, features, systems, devices, materials, methods and techniques described herein may, in some embodiments, be similar to any one or more of the embodiments, features, systems, devices, materials, methods and techniques described in the following:
U.S. application Ser. No. 16/048,167, filed on Jul. 27, 2018, titled “SYSTEMS AND METHODS FOR ASSISTING AND AUGMENTING SURGICAL PROCEDURES;”
U.S. application Ser. No. 16/242,877, filed on Jan. 8, 2019, titled “SYSTEMS AND METHODS OF ASSISTING A SURGEON WITH SCREW PLACEMENT DURING SPINAL SURGERY;”
U.S. application Ser. No. 16/207,116, filed on Dec. 1, 2018, titled “SYSTEMS AND METHODS FOR MULTI-PLANAR ORTHOPEDIC ALIGNMENT;”
U.S. application Ser. No. 16/352,699, filed on Mar. 13, 2019, titled “SYSTEMS AND METHODS FOR ORTHOPEDIC IMPLANT FIXATION;”
U.S. application Ser. No. 16/383,215, filed on Apr. 12, 2019, titled “SYSTEMS AND METHODS FOR ORTHOPEDIC IMPLANT FIXATION;”
U.S. application Ser. No. 16/569,494, filed on Sep. 12, 2019, titled “SYSTEMS AND METHODS FOR ORTHOPEDIC IMPLANTS;” and
U.S. application Ser. No. 16/699,447, filed Nov. 29, 2019, titled “SYSTEMS AND METHODS FOR ORTHOPEDIC IMPLANTS;”
U.S. application Ser. No. 16/735,222, filed Jan. 6, 2020, titled “PATIENT-SPECIFIC MEDICAL PROCEDURES AND DEVICES, AND ASSOCIATED SYSTEMS AND METHODS;”
U.S. application Ser. No. 16/987,113, filed Aug. 6, 2020, titled “PATIENT-SPECIFIC ARTIFICIAL DISCS, IMPLANTS AND ASSOCIATED SYSTEMS AND METHODS;”
U.S. application Ser. No. 16/990,810, filed Aug. 11, 2020, titled “LINKING PATIENT-SPECIFIC MEDICAL DEVICES WITH PATIENT-SPECIFIC DATA, AND ASSOCIATED SYSTEMS, DEVICES, AND METHODS;”
U.S. application Ser. No. 17/085,564, filed Oct. 30, 2020, titled “SYSTEMS AND METHODS FOR DESIGNING ORTHOPEDIC IMPLANTS BASED ON TISSUE CHARACTERISTICS;”
U.S. application Ser. No. 17/100,396, filed Nov. 20, 2020, titled “PATIENT-SPECIFIC VERTEBRAL IMPLANTS WITH POSITIONING FEATURES;”
U.S. application Ser. No. 17/342,439, filed Jun. 8, 2021, titled ““PATIENT-SPECIFIC MEDICAL PROCEDURES AND DEVICES, AND ASSOCIATED SYSTEMS AND METHODS;”
U.S. application Ser. No. 17/463,054, filed Aug. 31, 2021, titled “BLOCKCHAIN MANAGED MEDICAL IMPLANTS;”
U.S. application Ser. No. 17/518,524, filed Nov. 3, 2021, titled “PATIENT-SPECIFIC ARTHROPLASTY DEVICES AND ASSOCIATED SYSTEMS AND METHODS;”
U.S. application Ser. No. 17/531,417, filed Nov. 19, 2021, titled “PATIENT-SPECIFIC JIG FOR PERSONALIZED SURGERY;”
U.S. application Ser. No. 17/678,874, filed Feb. 23, 2022, titled “NON-FUNGIBLE TOKEN SYSTEMS AND METHODS FOR STORING AND ACCESSING HEALTHCARE DATA;”
U.S. application Ser. No. 17/835,777, filed Jun. 8, 2022, titled “PATIENT-SPECIFIC EXPANDABLE INTERVERTEBRAL IMPLANTS;”
U.S. application Ser. No. 17/842,242, filed Jun. 16, 2022, titled “PATIENT-SPECIFIC ANTERIOR PLATE IMPLANTS;”
U.S. application Ser. No. 17/851,487, filed Jun. 28, 2022, titled “PATIENT-SPECIFIC ADJUSTMENT OF SPINAL IMPLANTS, AND ASSOCIATED SYSTEMS AND METHODS;”
U.S. application Ser. No. 17/856,625, filed Jul. 1, 2022, titled “SPINAL IMPLANTS FOR MESH NETWORKS;”
U.S. application Ser. No. 17/867,621, filed Jul. 18, 2022, titled “PATIENT-SPECIFIC SACROILIAC IMPLANT, AND ASSOCIATED SYSTEMS AND METHODS;”
U.S. application Ser. No. 17/868,729, filed Jul. 19, 2022, titled “SYSTEMS FOR PREDICTING INTRAOPERATIVE PATIENT MOBILITY AND IDENTIFYING MOBILITY-RELATED SURGICAL STEPS;”
U.S. application Ser. No. 17/978,673, filed Nov. 1, 2022, titled “SPINAL IMPLANTS AND SURGICAL PROCEDURES WITH REDUCED SUBSIDENCE, AND ASSOCIATED SYSTEMS AND METHODS;”
U.S. application Ser. No. 17/978,746, filed Nov. 1, 2022, titled “PATIENT-SPECIFIC SPINAL INSTRUMENTS FOR IMPLANTING IMPLANTS AND DECOMPRESSION PROCEDURES;”
U.S. application Ser. No. 18/102,444, filed Jan. 27, 2023, titled “TECHNIQUES TO MAP THREE-DIMENSIONAL HUMAN ANATOMY DATA TO TWO-DIMENSIONAL HUMAN ANATOMY DATA;”
U.S. application Ser. No. 18/113,573, filed Feb. 24, 2023, titled “PATIENT-SPECIFIC IMPLANT DESIGN AND MANUFACTURING SYSTEM WITH A DIGITAL FILING CABINET;”
U.S. application Ser. No. 18/120,979, filed Mar. 13, 2023, titled “MULTI-STAGE PATIENT-SPECIFIC SURGICAL PLANS AND SYSTEMS AND METHODS FOR CREATING AND IMPLEMENTING THE SAME;”
U.S. application Ser. No. 17/951,085, filed Sep. 22, 2022, titled “SYSTEMS FOR MANUFACTURING AND PRE-OPERATIVE INSPECTING OF PATIENT-SPECIFIC IMPLANTS;”
U.S. Application No. 63/420,279, filed Oct. 28, 2022, titled “SYSTEMS AND METHODS FOR SELECTING, REVIEWING, MODIFYING, AND/OR APPROVING SURGICAL PLANS;”
U.S. Application No. 63/387,009, filed Dec. 12, 2022, titled “PATIENT-SPECIFIC IMPLANT DESIGN AND MANUFACTURING SYSTEM WITH A REGULATORY AND REIMBURSEMENT MANAGER;”
U.S. Application No. 63/436,860, filed Jan. 3, 2023, titled “PATIENT-SPECIFIC SPINAL FUSION DEVICES AND ASSOCIATED SYSTEMS AND METHODS;”
U.S. Application No. 63/437,966, filed Jan. 9, 2023, titled “SYSTEM FOR EDGE CASE PATHOLOGY IDENTIFICATION AND IMPLANT MANUFACTURING;”
U.S. Application No. 63/437,975, filed Jan. 9, 2023, titled “SYSTEM FOR MODELING PATIENT SPINAL CHANGES;”
U.S. Application No. 63/522,815, filed Jun. 23, 2023, titled “SYSTEMS AND METHODS FOR DIAGNOSING SPINAL CONDITIONS AND DETERMINING TREATMENT OF THE SAME;”
U.S. Application No. 63/530,427, filed Aug. 2, 2023, titled “MEDICAL DEVICE INSERTER INSTRUMENTS WITH RETRACTABLE COUPLING ELEMENTS AND METHODS OF USING THE SAME.”

All of the above-identified patents and applications are incorporated by reference in their entireties. In addition, the embodiments, features, systems, devices, materials, methods and techniques described herein may, in certain embodiments, be applied to or used in connection with any one or more of the embodiments, features, systems, devices, or other matter.

The ranges disclosed herein also encompass any and all overlap, sub-ranges, and combinations thereof. Language such as “up to,” “at least,” “greater than,” “less than,” “between,” or the like includes the number recited. Numbers preceded by a term such as “approximately,” “about,” and “substantially” as used herein include the recited numbers (e.g., about 10%=10%), and also represent an amount close to the stated amount that still performs a desired function or achieves a desired result. For example, the terms “approximately,” “about,” and “substantially” may refer to an amount that is within less than 10% of, within less than 5% of, within less than 1% of, within less than 0.1% of, and within less than 0.01% of the stated amount.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting.

What is claimed is:

1. An implant system comprising:
an intervertebral implant configured for implantation in a patient, the intervertebral implant including a superior patient-specific surface, an inferior patient-specific surface, and a main body configured to receive material configured to promote spinal fusion;
a first predicted post-operative three-dimensional representation and/or virtual model of patient anatomy associated with a first surgical procedure and including a first set of predicted post-operative spinal metrics; and
a second predicted post-operative three-dimensional representation and/or virtual model of patient anatomy associated with a second surgical procedure and including a second set of predicted post-operative spinal metrics,
wherein the intervertebral implant is configured to be implanted in conjunction with either the first surgical procedure or the second surgical procedure, and wherein the intervertebral implant is designed based at least in part on the corresponding predicted post-operative three-dimensional representation and/or virtual model.

2. The implant system of claim 1 wherein the superior patient-specific surface of the intervertebral implant matches a first vertebral surface of the corresponding post-operative three-dimensional representation and/or virtual model, and the inferior patient-specific surface of the intervertebral implant matches a second vertebral surface of the corresponding post-operative three-dimensional representation and/or virtual model.

3. The implant system of claim 1, further comprising one or more implants configured to be implanted in conjunction with the intervertebral implant, wherein the one or more implants are designed based at least in part on the corresponding post-operative three-dimensional representation and/or virtual model.

4. The implant system of claim 1 wherein the first predicted post-operative three-dimensional representation or virtual model is linked to an interactive surgical planner configured to receive at least one physician post-operative metric, wherein the first predicted post-operative three-dimensional representation or virtual model is configured to be modified to meet the at least one physician post-operative metric and to be measured for obtaining a modified first set of predicted post-operative spinal metrics based on the modified post-operative three-dimensional representation or virtual model.

5. The implant system of claim 1 wherein the first predicted post-operative three-dimensional representation or virtual model is linked to an account for the patient and configured for post-operative metric measuring, wherein the account includes simulated post-operative metrics for at least the first surgical procedure and the second surgical procedure.

6. The implant system of claim 1 wherein an account for the patient is linked to a surgical procedure being performed on the patient to enable real-time intra-operative viewing of simulated post-operative metrics for the surgical procedure.

7. The implant system of claim 1 wherein the first predicted post-operative three-dimensional representation and/or virtual model and the second predicted post-operative three-dimensional representation and/or virtual model include repositionable spinal anatomical elements.

8. The implant system of claim 7 wherein:
the first predicted post-operative three-dimensional representation and/or virtual model includes first model data for measuring the first set of predicted post-operative spinal metrics in a first predicted post-operative configuration associated with performance of the first surgical procedure, and
the second predicted post-operative three-dimensional representation and/or virtual model includes second model data for measuring the second set of predicted post-operative spinal metrics in a second predicted post-operative configuration associated with performance of the second surgical procedure.

9. The implant system of claim 8 wherein the first model data and/or the second model data include surface rendering data, measured patient metrics, and/or image data from images of the patient.

10. The implant system of claim 1, further comprising an interactive surgical plan configured to concurrently display:
the first predicted post-operative three-dimensional representation and/or virtual model of patient anatomy and the first set of predicted post-operative spinal metrics; and
the second predicted post-operative three-dimensional representation and/or virtual model of patient anatomy and the second set of predicted post-operative spinal metrics.

11. The implant system of claim 1, further comprising an interactive surgical plan configured to receive user input and to display dynamic comparisons of the first surgical procedure and the second surgical procedure based on user input.

12. The implant system of claim 1, further comprising an interactive surgical planner visually comparing multiple surgical plans with predicted outcome data.
